(19) United States
(12) Patent Application Publication
(10) Pub. No.: US 2010/0035926 A1
(43) Pub. Date: Feb. 11, 2010

(54) PYRAZOLE DERIVATIVES AS NON-STEROIDAL GLUCOCORTICOID RECEPTOR LIGANDS

(76) Inventors: Ian Baxter Campbell, Hertfordshire (GB); Diane Mary Coe, Hertfordshire (GB); Anthony William James Cooper, Hertfordshire (GB); Graham George Adam Inglis, Hertfordshire (GB); Haydn Terence Jones, Hertfordshire (GB); Steven Philip Keeling, Hertfordshire (GB); Simon John Fawcett MacDonald, Hertfordshire (GB); Philip Alan Skone, Hertfordshire (GB); Gordon Gad Weingarten, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/518,308

(22) PCT Filed: Dec. 19, 2007
(86) PCT No.: PCT/EP07/64167

§ 371 Date: Jun. 9, 2009

(30) Foreign Application Priority Data
Dec. 20, 2006 (GB) 062456.9
Jan. 3, 2007 (GB) 0700078.9

(51) Int. Cl. A61K 31/415; A61K 31/4439; A61K 31/4709; A61P 29/00; A61P 37/00; C07D 231/38; C07D 401/12

(52) U.S. Cl. 514/314; 514/407; 514/341; 548/371.7; 546/275.4; 546/165

(57) ABSTRACT

The present invention provides compounds of formula (I):

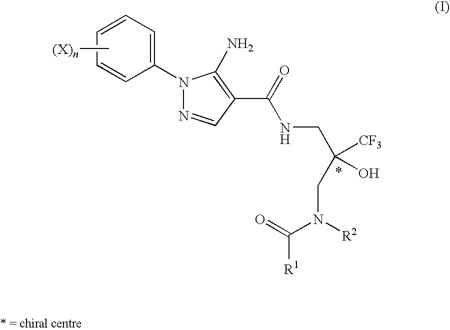

a process for their preparation, to pharmaceutical compositions comprising the compounds and the preparation of said compositions, to intermediates, and to use of the compounds for the manufacture of a medicament for therapeutic treatment, particularly for the treatment of inflammation.

The present invention relates to non-steroidal glucocorticoid receptor binding compounds and a process for their preparation, to pharmaceutical compositions comprising the compounds and the preparation of said compositions, to intermediates, and to use of the compounds for the manufacture of a medicament for therapeutic treatment, particularly for the treatment of inflammation.

Nuclear receptors are a class of structurally related proteins involved in the regulation of gene expression. The steroid hormone receptors are a subset of this family whose natural ligands typically comprise endogenous steroids such as estradiol (estrogen receptor), progesterone (progesterone receptor) and cortisol (glucocorticoid receptor). Man-made ligands to these receptors play an important role in human health, in particular the use of glucocorticoid agonists to treat a wide range of inflammatory conditions.

Current known glucocorticoids have proved useful in the treatment of inflammation, tissue rejection, auto-immunity, various malignancies, such as leukemias and lymphomas, Cushing's syndrome, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia and Little's syndrome.

Glucocorticoids are especially useful in disease states involving systemic inflammation such as inflammatory bowel disease, systemic lupus erythematosus, polyarteritis nodosa, Wegener's granulomatosis, giant cell arteritis, rheumatoid arthritis, osteoarthritis, seasonal rhinitis, allergic rhinitis, vasomotor rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis and cirrhosis. Glucocorticoids have also been used as immunostimulants and repressors and as wound healing and tissue repair agents.

Glucocorticoids have also found use in the treatment of diseases such as inflammatory scalp alopecia, panniculitis, psoriasis, discoid lupus erythemnatosus, inflamed cysts, atopic dermatitis, pyoderma gangrenosum, pemphigus vulgaris, bullous pemphigoid, systemic lupus erythematosus, dermatomyositis, herpes gestationis, eosinophilic fasciitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitis, erythema nodosum, acne, hirsutism, toxic epidermal necrolysis, erythema multiform and cutaneous T-cell lymphoma.

A number of conditions where a key component of the pathology is inflammation within the central nervous system (CNS) are currently treated with high doses of glucocorticoid agents. It is understood that these high doses are required primarily because the steroidal agents are actively removed from the brain by specific transporters, and therefore high systemic concentrations must be achieved in order to reach therapeutic doses within the CNS. Agents which showed a higher propensity to partition into the brain would allow these therapeutic concentrations to be achieved within the CNS with a significant reduction in the systemic glucocorticoid burden, resulting in an reduced risk from the known systemic effects of glucocorticoids (such as osteoporosis, diabetes, myopathy, skin thinning and weight gain).

Inflammatory or auto-immune conditions of the nervous system where such an approach may prove valuable include but are not limited to multiple sclerosis, cerebral vasculitis, neurosarcoidosis, Sjogren's syndrome, systemic lupus erythematosis, acute or chronic inflammatory polyradiculopathy, Alzheimer's disease, neoplastic diseases of the nervous system including meningioma, lymphoma and malignant meningitis, and trauma and infectious diseases of the nervous system such as tuberculosis. Other conditions include brain injury, for example post-infarction (stroke).

There remains a need to find further compounds which bind to the glucocorticoid receptor.

The present invention provides compounds of formula (I):

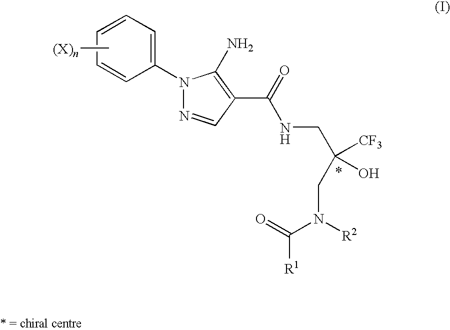

wherein
R¹ is selected from

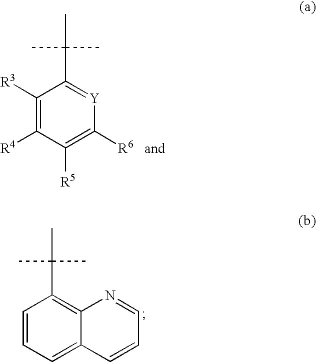

R² is selected from methyl, ethyl and 2-fluoroethyl;
R³, R⁴, R⁵ and R⁶ are each independently selected from hydrogen, fluorine, chlorine, —CF₃, —CHF₂, —OCHF₂ and —C(O)CH₃;
Y is selected from nitrogen and CH;
n is an integer selected from 0, 1 and 2,
when n is 1, X is selected from chlorine and fluorine, and
when n is 2, each X is fluorine;
and salts thereof (hereinafter “compounds of the invention”).

In a further embodiment, the present invention provides compounds of formula (IA):

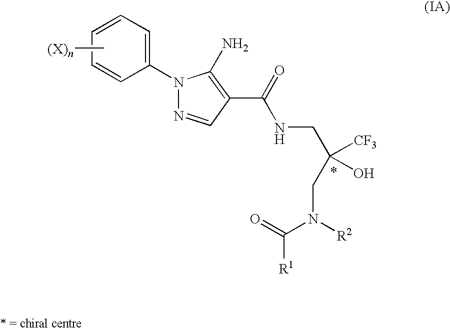

wherein
R¹ is selected from

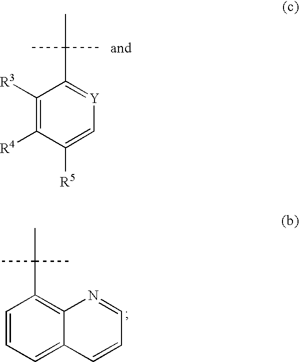

R² is selected from methyl, ethyl and 2-fluoroethyl;
R³, R⁴ and R⁵ are each independently selected from hydrogen, fluorine, chlorine, —CF₃, —CHF₂, —OCHF₂ and —C(O)CH₃;
Y is selected from nitrogen and CH;
n is an integer selected from 0, 1 and 2,
when n is 1, X is selected from chlorine and fluorine, and
when n is 2, each X is fluorine;
and salts thereof.

In one embodiment, R¹ is

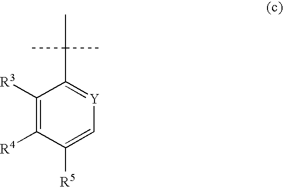

In a further embodiment R¹ is

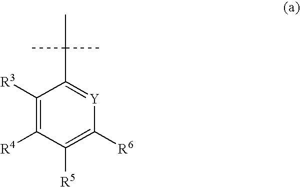

In one embodiment R² is selected from ethyl and 2-fluoroethyl. In another embodiment R² is ethyl. In another embodiment R² is 2-fluoroethyl. In a further embodiment R² is methyl.

In one embodiment R³ is selected from hydrogen, fluorine, chlorine, —CF₃, —OCHF₂ and —C(O)CH₃. In another embodiment R³ is selected from fluorine, chlorine, —CF₃, —OCHF₂ and —C(O)CH₃. In a further embodiment R³ is selected from hydrogen, fluorine and chlorine.

In one embodiment R⁴ is selected from hydrogen and fluorine. In another embodiment R⁴ is hydrogen. In a further embodiment R⁴ is fluorine.

In one embodiment R⁵ is hydrogen.

In one embodiment R⁶ is selected from hydrogen and chlorine. In a further embodiment R⁶ is hydrogen.

In one embodiment Y is nitrogen. In a further embodiment Y is CH.

In one embodiment n is 1. In another embodiment n is 2.

In one embodiment when n is 1, X is fluorine. In a further embodiment the fluorine is in the para position on the phenyl ring.

In one embodiment when n is 2, X is fluorine. In a further embodiment one fluorine is in the para position and the second fluorine is in the meta position on the phenyl ring.

It is to be understood that the present invention covers all combinations of substituent groups described hereinabove.

“Compounds of the invention” include each of examples 1 to 46 and salts thereof.

In one embodiment, the compound of formula (I) is:
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide;
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide (Enantiomer 1);
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide (Enantiomer 2);
5-amino-1-(4-fluorophenyl)-N-[3,3,3-trifluoro-2-({(2-fluoroethyl)[(2-fluorophenyl)carbonyl]amino}methyl)-2-hydroxypropyl]-1H-pyrazole-4-carboxamide;
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide;
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide (Enantiomer 1);
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide (Enantiomer 2);
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide;
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide (Enantiomer 1);
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide (Enantiomer 2);
5-amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide;
5-amino-1-(4-fluorophenyl)-N-(3,3,3-trifluoro-2-{[[(2-fluorophenyl)carbonyl](methyl)amino]methyl}-2-hydroxypropyl)-1H-pyrazole-4-carboxamide;
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide;
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide (Enantiomer 1);
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide (Enantiomer 2);
N-{2-[({[5-amino-1-(4-fluorophenyl)-1H-pyrazol-4-yl]carbonyl}amino)methyl]-3,3,3-trifluoro-2-hydroxypropyl}-N-ethyl-8-quinolinecarboxamide;
5-amino-N-{2-[(ethyl{[2-(trifluoromethyl)phenyl]carbonyl}amino)methyl]-3,3,3-trifluoro-2-hydroxypropyl}-1-(4-fluorophenyl)-1H-pyrazole-4-carboxamide;
5-amino-N-{2-[(ethyl{[2-(trifluoromethyl)phenyl]carbonyl}amino)methyl]-3,3,3-trifluoro-2-hydroxypropyl}-1-(4-fluorophenyl)-1H-pyrazole-4-carboxamide (Enantiomer 1);
5-amino-N-{2-[(ethyl{[2-(trifluoromethyl)phenyl]carbonyl}amino)methyl]-3,3,3-trifluoro-2-hydroxypropyl}-1-(4-fluorophenyl)-1H-pyrazole-4-carboxamide (Enantiomer 2);
5-amino-1-(4-fluorophenyl)-N-{3,3,3-trifluoro-2-[((2-fluoroethyl){[2-(trifluoromethyl)phenyl]carbonyl}amino)methyl]-2-hydroxypropyl}-1H-pyrazole-4-carboxamide;
5-amino-N-(2-{[({2-[(difluoromethyl)oxy]phenyl}carbonyl)(2-fluoroethyl)amino]methyl}-3,3,3-trifluoro-2-hydroxypropyl)-1-(4-fluorophenyl)-1H-pyrazole-4-carboxamide;
5-amino-N-(2-{[({2-[(difluoromethyl)oxy]phenyl}carbonyl)(ethyl)amino]methyl}-3,3,3-trifluoro-2-hydroxypropyl)-1-(4-fluorophenyl)-1H-pyrazole-4-carboxamide;
N-(2-{[[(2-acetylphenyl)carbonyl](ethyl)amino]methyl}-3,3,3-trifluoro-2-hydroxypropyl)-5-amino-1-(4-fluorophenyl)-1H-pyrazole-4-carboxamide;
5-amino-N-(2-{[ethyl(phenylcarbonyl)amino]methyl}-3,3,3-trifluoro-2-hydroxypropyl)-1-(4-fluorophenyl)-1H-pyrazole-4-carboxamide;
5-amino-N-(2-{[ethyl(phenylcarbonyl)amino]methyl}-3,3,3-trifluoro-2-hydroxypropyl)-1-(4-fluorophenyl)-1H-pyrazole-4-carboxamide (Enantiomer 1);
5-amino-N-(2-{[ethyl(phenylcarbonyl)amino]methyl}-3,3,3-trifluoro-2-hydroxypropyl)-1-(4-fluorophenyl)-1H-pyrazole-4-carboxamide (Enantiomer 2);
5-amino-N-(2-{[[(2-chlorophenyl)carbonyl](2-fluoroethyl)amino]methyl}-3,3,3-trifluoro-2-hydroxypropyl)-1-(4-fluorophenyl)-1H-pyrazole-4-carboxamide;
5-amino-N-(2-{[[(2-chlorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(3-chlorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(2-chloro-3-fluorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(4-chlorophenyl)carbonyl](ethyl)amino]methyl}-3,3,3-trifluoro-2-hydroxypropyl)-1-(4-fluorophenyl)-1H-pyrazole-4-carboxamide;
5-amino-N-{2-[(ethyl{[3-(trifluoromethyl)phenyl]carbonyl}amino)methyl]-3,3,3-trifluoro-2-hydroxypropyl}-1-(4-fluorophenyl)-1H-pyrazole-4-carboxamide;
5-amino-N-(2-{[[(3,4-difluorophenyl)carbonyl](ethyl)amino]methyl}-3,3,3-trifluoro-2-hydroxypropyl)-1-(4-fluorophenyl)-1H-pyrazole-4-carboxamide;
N-(2-{[[(3-acetylphenyl)carbonyl](ethyl)amino]methyl}-3,3,3-trifluoro-2-hydroxypropyl)-5-amino-1-(4-fluorophenyl)-1H-pyrazole-4-carboxamide;
5-amino-N-(2-{[[(2-chloro-4-fluorophenyl)carbonyl](ethyl)amino]methyl}-3,3,3-trifluoro-2-hydroxypropyl)-1-(4-fluorophenyl)-1H-pyrazole-4-carboxamide;
5-amino-1-(3,4-difluorophenyl)-N-(2-{[ethyl(phenylcarbonyl)amino]methyl}-3,3,3-trifluoro-2-hydroxypropyl)-1H-pyrazole-4-carboxamide;
5-amino-1-(4-fluorophenyl)-N-(3,3,3-trifluoro-2-hydroxy-2-{[methyl(phenylcarbonyl)amino]methyl}propyl)-1H-pyrazole-4-carboxamide;
5-amino-1-(4-fluorophenyl)-N-{3,3,3-trifluoro-2-hydroxy-2-[(methyl{[3-(trifluoromethyl)phenyl]carbonyl}amino)methyl]propyl}-1H-pyrazole-4-carboxamide;
N-(2-{[[(2-acetylphenyl)carbonyl](methyl)amino]methyl}-3,3,3-trifluoro-2-hydroxypropyl)-5-amino-1-(4-fluorophenyl)-1H-pyrazole-4-carboxamide;
5-amino-1-(4-fluorophenyl)-N-{3,3,3-trifluoro-2-hydroxy-2-[(methyl{[2-(trifluoromethyl)phenyl]carbonyl}amino)methyl]propyl}-1H-pyrazole-4-carboxamide;
N-{2-[({[5-amino-1-(4-fluorophenyl)-1H-pyrazol-4-yl]carbonyl}amino)methyl]-3,3,3-trifluoro-2-hydroxypropyl}-3-fluoro-N-methyl-2-pyridinecarboxamide;
N-{2-[({[5-amino-1-(4-fluorophenyl)-1H-pyrazol-4-yl]carbonyl}amino)methyl]-3,3,3-trifluoro-2-hydroxypropyl}-3,5-dichloro-N-methyl-2-pyridinecarboxamide;
N-{2-[({[5-amino-1-(4-fluorophenyl)-1H-pyrazol-4-yl]carbonyl}amino)methyl]-3,3,3-trifluoro-2-hydroxypropyl}-3,5-difluoro-N-methyl-2-pyridinecarboxamide; or
   a salt thereof.

In another embodiment the compound of formula (I) is:
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide (Enantiomer 2);
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide (Enantiomer 1);
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide;
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide (Enantiomer 2);
5-amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide (Enantiomer 1);
5-amino-N-{2-[(ethyl{[2-(trifluoromethyl)phenyl]carbonyl}amino)methyl]-3,3,3-trifluoro-2-hydroxypropyl}-1-(4-fluorophenyl)-1H-pyrazole-4-carboxamide;
5-amino-N-{2-[(ethyl{[2-(trifluoromethyl)phenyl]carbonyl}amino)methyl]-3,3,3-trifluoro-2-hydroxypropyl}-1-(4-fluorophenyl)-1H-pyrazole-4-carboxamide (Enantiomer 2);
5-amino-1-(4-fluorophenyl)-N-{3,3,3-trifluoro-2-[((2-fluoroethyl){[2-(trifluoromethyl)phenyl]carbonyl}amino)methyl]-2-hydroxypropyl}-1H-pyrazole-4-carboxamide;
5-amino-N-(2-{[({2-[(difluoromethyl)oxy]phenyl}carbonyl)(ethyl)amino]methyl}-3,3,3-trifluoro-2-hydroxypropyl)-1-(4-fluorophenyl)-1H-pyrazole-4-carboxamide;
N-(2-{[[(2-acetylphenyl)carbonyl](ethyl)amino]methyl}-3,3,3-trifluoro-2-hydroxypropyl)-5-amino-1-(4-fluorophenyl)-1H-pyrazole-4-carboxamide;
5-amino-N-(2-{[[(2-chlorophenyl)carbonyl](ethyl)amino]methyl}-3,3,3-trifluoro-2-hydroxypropyl)-1-(4-fluorophenyl)-1H-pyrazole-4-carboxamide; or
   a salt thereof.

In another embodiment the compound of formula (I) is:
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide;
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide (Enantiomer 2);
5-amino-1-(4-fluorophenyl)-N-[3,3,3-trifluoro-2-({(2-fluoroethyl)[(2-fluorophenyl)carbonyl]amino}methyl)-2-hydroxypropyl]-1H-pyrazole-4-carboxamide;
5-amino-1-(4-fluorophenyl)-N-[3,3,3-trifluoro-2-({(2-fluoroethyl)[(2-fluorophenyl)carbonyl]amino}methyl)-2-hydroxypropyl]-1H-pyrazole-4-carboxamide (Enantiomer 2);
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide;
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide (Enantiomer 1);
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide;
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide (Enantiomer 2);
5-amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide (Enantiomer 1);
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide;
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide (Enantiomer 1);
N-{2-[({[5-amino-1-(4-fluorophenyl)-1H-pyrazol-4-yl]carbonyl}amino)methyl]-3,3,3-trifluoro-2-hydroxypropyl}-N-ethyl-8-quinolinecarboxamide;
N-{2-[({[5-amino-1-(4-fluorophenyl)-1H-pyrazol-4-yl]carbonyl}amino)methyl]-3,3,3-trifluoro-2-hydroxypropyl}-N-ethyl-8-quinolinecarboxamide (Enantiomer 1);
5-amino-N-(2-{[[(2-chlorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(2-chlorophenyl)carbonyl](ethyl)amino]methyl}-3,3,3-trifluoro-2-hydroxypropyl)-1-(4-fluorophenyl)-1H-pyrazole-4-carboxamide (Enantiomer 1);
5-amino-1-(3,4-difluorophenyl)-N-(2-{[ethyl(phenylcarbonyl)amino]methyl}-3,3,3-trifluoro-2-hydroxypropyl)-1H-pyrazole-4-carboxamide; or
   a salt thereof.

In a further embodiment the compound of formula (I) is:
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide (Enantiomer 2);
5-amino-1-(4-fluorophenyl)-N-[3,3,3-trifluoro-2-({(2-fluoroethyl)[(2-fluorophenyl)carbonyl]amino}methyl)-2-hydroxypropyl]-1H-pyrazole-4-carboxamide (Enantiomer 2);
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide (Enantiomer 1);
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide (Enantiomer 2);
5-amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide (Enantiomer 1);
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide (Enantiomer 1);
N-{2-[({[5-amino-1-(4-fluorophenyl)-1H-pyrazol-4-yl]carbonyl}amino)methyl]-3,3,3-trifluoro-2-hydroxypropyl}-N-ethyl-8-quinolinecarboxamide (Enantiomer 1);
5-amino-N-(2-{[[(2-chlorophenyl)carbonyl](ethyl)amino]methyl}-3,3,3-trifluoro-2-hydroxypropyl)-1-(4-fluorophenyl)-1H-pyrazole-4-carboxamide (Enantiomer 1);
5-amino-1-(3,4-difluorophenyl)-N-(2-{[ethyl(phenylcarbonyl)amino]methyl}-3,3,3-trifluoro-2-hydroxypropyl)-1H-pyrazole-4-carboxamide; or
   a salt thereof.

Also included within the scope of the “compounds of the invention” are all solvates, hydrates, complexes and polymorphic forms of the compounds of formula (I) and salts thereof.

The compounds of formula (I) each contain a chiral centre and there are two possible enantiomers of each compound of formula (I).

The terms Enantiomer 1 and Enantiomer 2 are used herein to refer to the enantiomers of a compound of formula (I), based on the order of their elution using the chiral chromatography methodology described herein. Enantiomer 1 refers to the first enantiomer to elute, and Enantiomer 2 refers to the second enantiomer to elute.

A mixture of enantiomers, such as a racemic mixture, may be preferred. Thus, in one embodiment of the invention the compound of formula (I) is the racemic mixture (the racemate).

Alternatively, a single enantiomer may be preferred, for example Enantiomer 1. Thus, in one embodiment of the invention the compound of formula (I) is Enantiomer 1. In a further embodiment of the invention the compound of formula (I) is Enantiomer 2.

It will be appreciated by those skilled in the art that as rotation of the C(O)—R¹ bond becomes less facile due to ortho substitution on the aromatic ring, atropisomerism may be observed thus creating the possibility of four isomers namely Atropisomer 1, Enantiomer 1 (A1E1); Atropisomer 1, Enantiomer 2 (A1E2); Atropisomer 2, Enantiomer 1 (A2E1); and Atropisomer 2, Enantiomer 2 (A2E2). Any comment relating to the biological activity of an isomer or stereoisomer should be taken to include these atropisomers. It will be appreciated by those skilled in the art that where there is a non 1:1 ratio of atropisomers, that this ratio can change depending on the half life of interconversion.

It will be further appreciated by those skilled in the art that, for compounds of formula (I) wherein rotation is restricted around the C(O)—NR² bond due to substitution of the amide, for example when R² is ethyl or 2-fluoroethyl, rotamers may be observed. Any comment relating to the biological activity of an isomer or stereoisomer should be taken to include these rotamers. It will be appreciated by those skilled in the art that there may not be a 1:1 ratio of rotamers as the ratio can change depending on the half life of interconversion.

The terms “stereoisomer” and “isomer” as used herein encompass enantiomer, atropisomer and/or rotamer.

At least one of the possible stereoisomers of each of the compounds of formula (I) may bind to the glucocorticoid receptor. Further, it appears that at least one of the possible stereoisomers of each of the compounds of formula (I) may have glucocorticoid receptor agonist activity.

Accordingly, at least one of the possible stereoisomers of each compound of formula (I) modulates the glucocorticoid receptor. The term “modulator” as used herein refers to a compound which binds to the glucocorticoid receptor and acts as either an agonist, a partial agonist or an antagonist of the glucocorticoid receptor.

The compounds of the invention may provide agonism of the glucocorticoid receptor. Certain compounds of the invention may show a propensity to partition into the brain. Agents which show a higher propensity to partition into the brain may allow therapeutic concentrations to be achieved within the CNS with a significant reduction in the systemic glucocorticoid burden, resulting in an reduced risk from the known systemic effects of glucocorticoids (such as osteoporosis, diabetes, myopathy, skin thinning and weight gain).

It will be appreciated by those skilled in the art that at least one isomer (e.g. one enantiomer of the racemate) may have the described activity. The other isomers may have similar activity, less activity, no activity or may have some antagonist activity in a functional assay.

One embodiment of the invention embraces compounds of formula (I) and salts and solvates thereof. Another embodiment of the invention embraces compounds of formula (I) and salts thereof. Another embodiment of the invention embraces compounds of formula (I) and solvates thereof. A further embodiment of the invention embraces compounds of formula (I) as the free base.

Salts of the compounds of formula (I) which are suitable for use in medicine are those wherein the counter-ion or associated solvent is pharmaceutically acceptable. However, salts having non-pharmaceutically acceptable counter-ions or associated solvents are within the scope of the present invention, for example, for use as intermediates in the preparation of other compounds of formula (I) and their pharmaceutically acceptable salts.

Suitable salts according to the invention include those formed with both organic and inorganic acids or bases. Pharmaceutically acceptable acid addition salts may include those formed from hydrochloric, hydrobromic, sulphuric, phosphoric, trifluoroacetic, sulphamic, sulphanilic, methanesulphonic, ethanesulphonic and arylsulphonic (for example p-toluenesulphonic, benzenesulphonic, naphthalenesulphonic or naphthalenedisulphonic) acids. Pharmaceutically acceptable base salts may include alkali metal salts such as those of sodium and potassium and alkaline earth metal salts such as those of calcium. Hence, one embodiment of the invention embraces compounds of formula (I) and pharmaceutically acceptable salts thereof.

The compounds of the invention may have the ability to crystallise in more than one form. This is a characteristic known as polymorphism, and it is understood that such polymorphic forms (“polymorphs”) are within the scope of the present invention. Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

The compounds of the invention are expected to have potentially beneficial anti-inflammatory effects, particularly upon oral administration, demonstrated by, for example, their ability to bind to the glucocorticoid receptor and to elicit a response via that receptor. Hence, the compounds of the invention may be of use in the treatment of inflammatory and/or auto-immune disorders.

The compounds of the invention may also have potentially beneficial anti-allergic effects, particularly upon oral administration, demonstrated by, for example, their ability to bind to the glucocorticoid receptor and to elicit a response via that receptor. Hence, the compounds of the invention may be of use in the treatment of allergic disorders.

Examples of disease states in which the compounds of the invention are expected to have utility include multiple sclerosis, cerebral vasculitis, neurosarcoidosis, Sjogren's syndrome, systemic lupus erythematosis, acute or chronic inflammatory polyradiculopathy, Alzheimer's disease, neoplastic diseases of the nervous system including meningioma, lymphoma and malignant meningitis, and trauma and infectious diseases of the nervous system such as tuberculosis. Other conditions include brain injury, for example post-infarction (stroke).

Examples of further disease states associated with glucocorticoid receptor activity include skin diseases such as eczema, psoriasis, allergic dermatitis, neurodermatitis, pruritis, exfoliative dermatitis, pemphigus and hypersensitivity reactions; inflammatory conditions of the nose, throat or lungs such as asthma (including allergen-induced asthmatic reactions), rhinitis (including seasonal (hayfever), allergic and vasomotor), nasal polyps, chronic obstructive pulmonary disease (COPD), interstitial lung disease, and fibrosis; inflammatory bowel conditions such as ulcerative colitis and Crohn's disease; auto-immune diseases such as rheumatoid arthritis, temporal arteritis, polyarteritis nodosa, polymyositis, ankylosing spondylitis, sarcoidosis, autoimmune hepatitis; cancers such as acute and lymphatic leukaemia, myeloma, lymphoma; nephritic syndrome; septic shock; adrenal insufficiency; ophthalmic inflammation and allergic conjunctivitis; obesity; diabetes; chronic inflammatory pain including musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea; psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof. Compounds having glucocorticoid receptor activity may also have utility in inducing suppression of the immune system during organ transplantation, in acute transplant reject, angioedema of the upper respiratory tract and anaphylactic shock.

It will be appreciated by those skilled in the art that reference herein to treatment extends to prophylaxis as well as the treatment of established conditions.

As mentioned above, compounds of the invention are expected to be of use in human or veterinary medicine, in particular as anti-inflammatory agents.

There is thus provided as a further aspect of the invention a compound of formula (I) or a pharmaceutically acceptable salt thereof for use in human or veterinary medicine, particularly in the treatment of patients with inflammatory and/or allergic and/or auto-immune conditions.

There is thus provided as a further aspect of the invention a compound of formula (I) or a pharmaceutically acceptable salt thereof for use in human or veterinary medicine, particularly in the treatment of patients with inflammatory and/or auto-immune conditions, such as conditions involving inflammation within the central nervous system.

In another aspect of the invention there is provided a compound of formula (I) or a pharmaceutically acceptable salt thereof for use in the treatment of patients with multiple sclerosis, cerebral vasculitis, neurosarcoidosis, Sjogren's syndrome, systemic lupus erythematosis, acute or chronic inflammatory polyradiculopathy, Alzheimer's disease, neoplastic diseases of the nervous system including meningioma, lymphoma and malignant meningitis, trauma or infectious diseases of the nervous system such as tuberculosis, or brain injury such as post-infarction (stroke).

In another aspect of the invention there is provided a compound of formula (I) or a pharmaceutically acceptable salt thereof for use in the treatment of patients with neurosarcoidosis.

In another aspect of the invention there is provided a compound of formula (I) or a pharmaceutically acceptable salt thereof for use in the treatment of patients with rheumatoid arthritis, asthma, COPD, allergy and/or rhinitis.

In another aspect of the invention there is provided a compound of formula (I) or a pharmaceutically acceptable salt thereof for use in the treatment of patients with skin disease such as eczema, psoriasis, allergic dermatitis, neurodermatitis, pruritis and/or hypersensitivity reactions.

According to another aspect of the invention, there is provided the use of a compound of formula (I) or a pharmaceutically acceptable salt thereof for the manufacture of a medicament for the treatment of patients with inflammatory and/or allergic and/or auto-immune conditions.

According to another aspect of the invention, there is provided the use of a compound of formula (I) or a pharmaceutically acceptable salt thereof for the manufacture of a medicament for the treatment of patients with inflammatory and/or auto-immune conditions, such as conditions involving inflammation within the central nervous system.

According to another aspect of the invention, there is provided the use of a compound of formula (I) or a pharmaceutically acceptable salt thereof for the manufacture of a medicament for the treatment of patients with multiple sclerosis, cerebral vasculitis, neurosarcoidosis, Sjogren's syndrome, systemic lupus erythematosis, acute or chronic inflammatory polyradiculopathy, Alzheimer's disease, neoplastic diseases of the nervous system including meningioma, lymphoma and malignant meningitis, trauma or infectious diseases of the nervous system such as tuberculosis, or brain injury such as post-infarction (stroke).

According to yet another aspect of the invention there is provided the use of a compound of formula (I) or a pharmaceutically acceptable salt thereof for the manufacture of a medicament for the treatment of patients with neurosarcoidosis.

According to yet another aspect of the invention there is provided the use of a compound of formula (I) or a pharmaceutically acceptable salt thereof for the manufacture of a medicament for the treatment of patients with rheumatoid arthritis, asthma, COPD, allergy and/or rhinitis.

According to yet another aspect of the invention there is provided the use of a compound of formula (I) or a pharmaceutically acceptable salt thereof for the manufacture of a medicament for the treatment of patients with skin disease such as eczema, psoriasis, allergic dermatitis, neurodermatitis, pruritis and/or hypersensitivity reactions.

In a further or alternative aspect, there is provided a method for the treatment of a human or animal subject with an inflammatory and/or allergic and/or auto-immune condition, which method comprises administering to said human or animal subject an effective amount of a compound of formula (I) or a pharmaceutically acceptable salt thereof.

In a further or alternative aspect, there is provided a method for the treatment of a human or animal subject with an inflammatory and/or auto-immune condition, which method comprises administering to said human or animal subject an effective amount of a compound of formula (I) or a pharmaceutically acceptable salt thereof.

In a further or alternative aspect, there is provided a method for the treatment of a human or animal subject with multiple sclerosis, cerebral vasculitis, neurosarcoidosis, Sjogren's syndrome, systemic lupus erythematosis, acute or chronic inflammatory polyradiculopathy, Alzheimer's disease, neoplastic diseases of the nervous system including meningioma, lymphoma and malignant meningitis, trauma or infectious diseases of the nervous system such as tuberculosis, or brain injury such as post-infarction (stroke), which method comprises administering to said human or animal subject an effective amount of a compound of formula (I) or a pharmaceutically acceptable salt thereof.

In yet a further or alternative aspect there is provided a method for the treatment of a human or animal subject with neurosarcoidosis, which method comprises administering to said human or animal subject an effective amount of a compound of formula (I) or a pharmaceutically acceptable salt thereof.

In yet a further or alternative aspect there is provided a method for the treatment of a human or animal subject with rheumatoid arthritis, asthma, COPD, allergy and/or rhinitis, which method comprises administering to said human or animal subject an effective amount of a compound of formula (I) or a pharmaceutically acceptable salt thereof.

In yet a further or alternative aspect there is provided a method for the treatment of a human or animal subject with skin disease such as eczema, psoriasis, allergic dermatitis, neurodermatitis, pruritis and/or hypersensitivity reactions, which method comprises administering to said human or animal subject an effective amount of a compound of formula (I) or a pharmaceutically acceptable salt thereof.

The compounds of the invention may be formulated for administration in any convenient way, and the invention therefore also includes within its scope pharmaceutical compositions comprising a compound of formula (I) or a pharmaceutically acceptable salt thereof together, if desirable, in admixture with one or more physiologically acceptable diluents or carriers.

Examples of physiologically acceptable diluents or carriers include, but are not limited to, aqueous or non-aqueous vehicles, thickening agents, isotonicity adjusting agents, antioxidants and/or preservatives.

Further, there is provided a process for the preparation of such pharmaceutical compositions which comprises mixing the ingredients. A pharmaceutical composition comprising a compound of formula (I) or a pharmaceutically acceptable salt thereof may be prepared by, for example, admixture at ambient temperature and atmospheric pressure.

The compounds of formula (I) and pharmaceutically acceptable salts thereof may, for example, be formulated for oral, nasal, buccal, sublingual, parenteral, rectal administration or other topical administration.

For systemic administration the compounds of formula (I) and pharmaceutically acceptable salts thereof may, for example, be formulated in conventional manner for oral, parenteral or rectal administration. Formulations for oral administration include solutions, syrups, elixirs, powders, granules, tablets and capsules which typically contain conventional excipients such as binding agents, fillers, lubricants, disintegrants, wetting agents, suspending agents, emulsifying agents, preservatives, buffer salts, flavouring, colouring and/or sweetening agents as appropriate. Dosage unit forms may be preferred as described below.

For instance, for oral administration in the form of a tablet or capsule, the active drug component can be combined with an oral, non-toxic pharmaceutically acceptable inert carrier such as ethanol, glycerol, water and the like. The tablets may also contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Examples of excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Powders are prepared by comminuting the compound to a suitable fine size and mixing with a similarly comminuted pharmaceutical carrier such as an edible carbohydrate, as, for example, starch or mannitol. Flavouring, preservative, dispersing and colouring agent can also be present.

Capsules can be made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of formula (I) and pharmaceutically acceptable salts thereof can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavour additives such as peppermint oil or saccharin, and the like can also be added. Where appropriate, dosage unit formulations for oral administration can be microencapsulated. The formulation can also be prepared to prolong or sustain the release as for example by coating or embedding particulate material in polymers, wax or the like.

The compounds of formula (I) or a pharmaceutically acceptable salt thereof can also be administered in the form of liposome emulsion delivery systems, such as small unilamellar vesicles, large unilamellar vesicles and multilamellar vesicles. Liposomes can be formed from a variety of phospholipids, such as cholesterol, stearylamine or phosphatidylcholines.

In one embodiment the compound of formula (I) or a pharmaceutically acceptable salt thereof is in the form of a tablet or capsule for oral administration for the treatment of neurosarcoidosis.

In one embodiment the compound of formula (I) or a pharmaceutically acceptable salt thereof is in the form of a solution, syrup or elixir for oral administration for the treatment of neurosarcoidosis.

Topical administration as used herein, includes administration by insufflation and inhalation. Examples of various types of preparation for topical administration include ointments, lotions, creams, gels, foams, preparations for delivery by transdermal patches, powders, sprays, aerosols, capsules or cartridges for use in an inhaler or insufflator or drops (e.g. eye or nose drops), solutions/suspensions for nebulisation, suppositories, pessaries, retention enemas and chewable or suckable tablets or pellets (e.g. for the treatment of aphthous ulcers) or liposome or microencapsulation preparations.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

The compounds of formula (I) and/or pharmaceutically acceptable salts thereof may be formulated for intransal delivery. According to one aspect of the invention there is provided a pharmaceutical composition comprising an aqueous suspension/solution of a compound of formula (I) and/or a pharmaceutically acceptable salt thereof and optionally; one or more suspending agents; one or more preservatives; one or more wetting agents; a buffer; one or more isotonicity adjusting agents; and one or more taste-masking agents. Compositions suitable for intranasal administration may optionally further contain other excipients, such as antioxidants (for example metabisulphite).

Examples of suspending agents include cellulose, carboxymethylcellulose, veegum, tragacanth, bentonite, methylcellulose and polyethylene glycols. In one embodiment the suspending agent will be microcrystalline cellulose and carboxy methylcellulose sodium, for example used as the branded product Avicel RC591 (which typically contains 87-91% microcrystalline cellulose and 9-13% carboxy methylcellulose sodium) or Avicel CL611.

For stability purposes, the composition of the present invention may be protected from microbial contamination and growth by inclusion of a preservative. Examples of pharmaceutically acceptable anti-microbial agents or preservatives that can be used in the composition include quaternary ammonium compounds (for example benzalkonium chloride, benzethonium chloride, cetrimide, cetylpyridinium chloride and myristyl picolinium chloride), alcoholic agents (for example chlorobutanol, phenylethyl alcohol and benzyl alcohol), antibacterial esters (for example esters of para-hydroxybenzoic acid), chelating agents such as disodium edetate (EDTA), and other anti-microbial agents such as chlorhexidine (for example in the form of the acetate or gluconate), potassium sorbate, chlorocresol, sorbic acid and its salts, polymyxin, methylparaben and propylparaben.

It will be appreciated that any agent which is effective in wetting the particles and which is pharmaceutically acceptable can be used. Examples of wetting agents that can be used are fatty alcohols, esters and ethers. In one embodiment the wetting agent is a hydrophilic, non-ionic surfactant, for example polyoxyethylene (20) sorbitan monooleate (supplied as the branded product Polysorbate 80).

Examples of buffer substances include citric acid/sodium hydrogensulphate borate buffers, citric acid/citrate buffers, phosphates (sodium hydrogenorthophosphate, disodium hydrogenphosphate), trometamol or equivalent conventional buffers in order to adjust the pH value of the composition.

The presence of an isotonicity adjusting agent is to achieve isotonicity with body fluids, for example fluids of the nasal cavity, resulting in reduced levels of irritancy associated with many nasal compositions. Examples of suitable isotonicity adjusting agents are glucose, glycerine, sorbitol, sodium chloride, dextrose and calcium chloride. In one embodiment the isotonicity adjusting agent may be dextrose, for example, anhydrous dextrose.

Examples of taste-masking agents include sucralose, sucrose, saccharin or a salt thereof, fructose, dextrose, corn syrup, aspartame, acesulfame-K, xylitol, sorbitol, erythritol, ammonium glycyrrhizinate, thaumatin, neotame, mannitol, menthol, eucalyptus oil, camphor, a natural flavouring agent, an artificial flavouring agent, and combinations thereof. In one embodiment the taste-masking agent is sucralose and/or menthol.

Compositions for administration topically to the nose or lung for example, for the treatment of rhinitis, include pressurised aerosol compositions and aqueous compositions delivered to the nasal cavities by pressurised pump. Compositions which are non-pressurised and adapted to be administered topically to the nasal cavity are of particular interest. Suitable compositions contain water as the diluent or carrier for this purpose. Aqueous compositions for administration to the lung or nose may be provided with conventional excipients such as buffering agents, tonicity modifying agents and the like. Aqueous compositions may also be administered to the nose by nebulisation.

A fluid dispenser may typically be used to deliver a fluid composition to the nasal cavities. The fluid composition may be aqueous or non-aqueous, but typically aqueous. Such a fluid dispenser may have a dispensing nozzle or dispensing orifice through which a metered dose of the fluid composition is dispensed upon the application of a user-applied force to a pump mechanism of the fluid dispenser. Such fluid dispensers are generally provided with a reservoir of multiple metered doses of the fluid composition, the doses being dispensable upon sequential pump actuations. The dispensing nozzle or orifice may be configured for insertion into the nostrils of the user for spray dispensing of the fluid composition into the nasal cavity. A fluid dispenser of the aforementioned type is described and illustrated in WO05/044354 the entire content of which is hereby incorporated herein by reference. The dispenser has a housing which houses a fluid discharge device having a compression pump mounted on a container for containing a fluid composition. The housing has at least one finger-operable side lever which is movable inwardly with respect to the housing to cam the container upwardly in the housing to cause the pump to compress and pump a metered dose of the composition out of a pump stem through a nasal nozzle of the housing. In one embodiment, the fluid dispenser is of the general type illustrated in FIGS. 30-40 of WO05/044354.

In one embodiment, there is provided an intranasal composition comprising a compound of formula (I) or a pharmaceutically acceptable salt thereof. In another embodiment, such an intranasal composition is benzalkonium chloride-free.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral administration to humans, the daily dosage level of the agent may be in single or divided doses.

For systemic administration the daily dose as employed for adult human treatment will range from 0.05-100 mg/kg body weight, preferably 0.1-60 mg/kg body weight, which may be administered in 1 to 4 daily doses, for example, depending on the route of administration and the condition of the patient. When the composition comprises dosage units, each unit will preferably contain 1 mg to 1 g of active ingredient. The duration of treatment will be dictated by the rate of response rather than by arbitrary numbers of days.

The compounds of the invention may in general be given by internal administration in cases wherein systemic glucocorticoid receptor agonist therapy is indicated.

Slow release or enteric coated formulations may be advantageous, particularly for the treatment of inflammatory bowel disorders.

In one embodiment, the compounds of the invention will be administered orally.

The compounds and pharmaceutical compositions according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents such as antibiotics or antivirals, or antihistamines. The invention thus provides, in a further aspect, a combination comprising a compound of formula (I) or a pharmaceutically acceptable salt thereof together with one or more other therapeutically active agents, for example selected from an anti-inflammatory agent such as a corticosteroid or an NSAID, an anticholinergic agent, a β₂-adrenoreceptor agonist, an antiinfective agent such as an antibiotic or an antiviral, or an antihistamine. One embodiment of the invention encompasses combinations comprising a compound of formula (I) or a pharmaceutically acceptable salt thereof together with a β₂-adrenoreceptor agonist, and/or an anticholinergic, and/or a PDE-4 inhibitor, and/or an antihistamine.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

Examples of β₂-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single diastereomer such as the R,R-diastereomer), salmefamol, fenoterol, carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment the β₂-adrenoreceptor agonists are long-acting β₂-adrenoreceptor agonists, for example, compounds which provide effective bronchodilation for about 12 hours or longer.

Other β₂-adrenoreceptor agonists include those described in WO02/066422, WO02/070490, WO02/076933, WO03/024439, WO03/072539, WO03/091204, WO04/016578, WO04/022547, WO04/037807, WO04/037773, WO04/037768, WO04/039762, WO04/039766, WO01/42193 and WO03/042160.

Examples of β₂-adrenoreceptor agonists include:
3-(4-{[6-({(2R)-2-hydroxy-2-[4-hydroxy-3-(hydroxymethyl)phenyl]ethyl}amino) hexyl]oxy}butyl)benzenesulfonamide;
3-(3-{[7-({(2R)-2-hydroxy-2-[4-hydroxy-3-hydroxymethyl)phenyl]ethyl}-amino) heptyl]oxy}propyl)benzenesulfonamide;
4-{(1R)-2-[(6-{2-[(2,6-dichlorobenzyl) oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Examples of corticosteroids which may be used in combination with a compound of formula (I) or a pharmaceutically acceptable salt thereof are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. In one embodiment corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO02/088167, WO02/100879, WO02/12265, WO02/12266, WO05/005451, WO05/005452, WO06/072599 and WO06/072600.

Non-steroidal compounds having glucocorticoid agonism that may possess selectivity for transrepression over transactivation and that may be useful in combination therapy include those covered in the following published patent applications and patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277, WO06/000401, WO06/000398, WO06/015870, WO06/108699, WO07/000,334 and WO07/054,294.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, WO02/50021, WO95/34534 and WO99/62875. Examples of CCR3 inhibitors include those disclosed in WO02/26722.

In one embodiment the invention provides the use of the compounds of formula (I) or a pharmaceutically acceptable salt thereof in combination with a phosphodiesterase 4 (PDE4) inhibitor, especially in the case of a composition adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4. Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd).

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Other anticholinergic agents include compounds which are disclosed in U.S. patent application 60/487,981 including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in U.S. patent application 60/511,009 including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzenesulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methanesulfonamide; and/or
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and/or
(endo)-3-{2,2-diphenyl-3-[(1-phenyl-methanoyl)-amino]-propyl}-8,8-dimethyl-8-azonia-bicyclo[3.2.1]octane bromide.

In one embodiment the invention provides a combination comprising a compound of formula (I) or a pharmaceutically acceptable salt thereof, together with an H1 antagonist. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I) or a pharmaceutically acceptable salt thereof, together with an H3 antagonist (and/or inverse agonist). Examples of H3 antagonists include, for example, those compounds disclosed in WO2004/035556 and in WO2006/045416. Other histamine receptor antagonists which may be used in combination with a compound of formula (I) or a pharmaceutically acceptable salt thereof, include antagonists (and/or inverse agonists) of the H4 receptor, for example, the compounds disclosed in Jablonowski et al., J. Med. Chem. 46:3957-3960 (2003).

The invention thus provides, in another aspect, a combination comprising a compound of formula (I) or a pharmaceutically acceptable salt thereof together with a PDE4 inhibitor.

The invention thus provides, in another aspect, a combination comprising a compound of formula (I) or a pharmaceutically acceptable salt thereof together with a β₂-adrenoreceptor agonist.

The invention thus provides, in another aspect, a combination comprising a compound of formula (I) or a pharmaceutically acceptable salt thereof together with a corticosteroid.

The invention thus provides, in another aspect, a combination comprising a compound of formula (I) or a pharmaceutically acceptable salt thereof together with another non-steroidal GR agonist.

The invention thus provides, in another aspect, a combination comprising a compound of formula (I) or a pharmaceutically acceptable salt thereof together with an anticholinergic.

The invention thus provides, in another aspect, a combination comprising a compound of formula (I) or a pharmaceutically acceptable salt thereof together with an antihistamine.

The invention thus provides, in a further aspect, a combination comprising a compound of formula (I) or a pharmaceutically acceptable salt thereof together with a PDE4 inhibitor and a β₂-adrenoreceptor agonist.

The invention thus provides, in a further aspect, a combination comprising a compound of formula (I) or a pharmaceutically acceptable salt thereof together with an anticholinergic and a PDE-4 inhibitor.

The individual compounds of such combinations may be administered either sequentially or simultaneously in separate or combined pharmaceutical compositions. In one embodiment, the individual compounds will be administered simultaneously in a combined pharmaceutical composition. Appropriate doses of known therapeutic agents will readily be appreciated by those skilled in the art.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The invention thus provides, in a further aspect, a pharmaceutical composition comprising a combination of a compound of formula (I) or a pharmaceutically acceptable salt thereof together with another therapeutically active agent.

The invention thus provides, in a further aspect, a pharmaceutical composition comprising a combination of a compound of formula (I) or a pharmaceutically acceptable salt thereof together with a PDE4 inhibitor.

The invention thus provides, in a further aspect, a pharmaceutical composition comprising a combination of a compound of formula (I) or a pharmaceutically acceptable salt thereof together with a β₂-adrenoreceptor agonist.

The invention thus provides, in a further aspect, a pharmaceutical composition comprising a combination of a compound of formula (I) or a pharmaceutically acceptable salt thereof together with a corticosteroid.

The invention thus provides, in a further aspect, a pharmaceutical composition comprising a combination of a compound of formula (I) or a pharmaceutically acceptable salt thereof together with another non-steroidal GR agonist.

The invention thus provides, in a further aspect, a pharmaceutical composition comprising a combination of a compound of formula (I) or a pharmaceutically acceptable salt thereof together with an anticholinergic.

The invention thus provides, in a further aspect, a pharmaceutical composition comprising a combination of a compound of formula (I) or a pharmaceutically acceptable salt thereof together with an antihistamine.

The invention thus provides, in a further aspect, a pharmaceutical composition comprising a compound of formula (I) or a pharmaceutically acceptable salt thereof together with a PDE4 inhibitor and a β₂-adrenoreceptor agonist.

The invention thus provides, in a further aspect, a pharmaceutical composition comprising a compound of formula (I) or a pharmaceutically acceptable salt thereof together with an anticholinergic and a PDE4 inhibitor.

A process according to the invention for the preparation of compounds of formula (I) comprises reaction of an amine of formula (II)

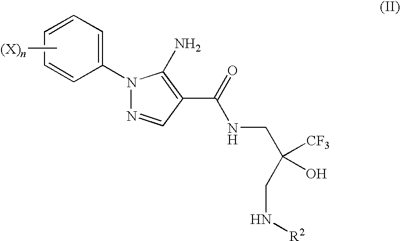

wherein R², X and n are as defined above for compounds of formula (I), with a compound of formula (III)

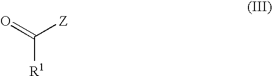

wherein R¹ is as defined above for compounds of formula (I) and Z is chlorine or hydroxy.

When Z is chlorine, the reaction may be carried out in a conventional organic solvent, for example tetrahydrofuran, in the presence of a base, for example potassium carbonate, triethylamine, pyridine or diisopropylethylamine. In one embodiment, the reaction is carried out in the presence of diisopropylethylamine. The reaction may be carried out at a temperature of from −10° C. to 100° C., for example at room temperature.

Alternatively, when Z is hydroxy, the reaction may be carried out in a conventional organic solvent, for example dimethylformamide, in the presence of a coupling agent such as those described in Tetrahedron 2005, 61, 10827, for example 0-(7-azabenzotriazol-1-yl)-N,N,N′,N′-tetramethyluronium hexafluorophosphate (HATU), and a base, for example triethylamine or diisopropylethylamine. In one embodiment, the reaction is carried out in the presence of diisopropylethylamine. The reaction may be carried out at a temperature of from −10° C. to 100° C., for example at room temperature.

Compounds of formula (II) wherein R² represents methyl, ethyl or 2-fluoroethyl may be prepared by reaction of a compound of formula (IV)

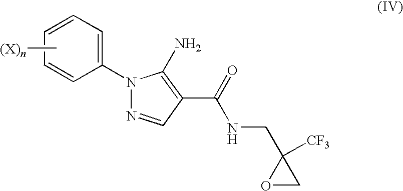

wherein X and n as defined above for compounds of formula (I),
with methylamine, ethylamine or 2-fluoroethylamine. The reaction may be carried out in a conventional organic solvent, for example acetonitrile or tetrahydrofuran, and at a temperature of from −10° C. to 100° C., for example at room temperature. 2-Fluoroethylamine may be produced in situ from 2-fluoroethylamine hydrochloride and a base, for example triethylamine.

A compound of formula (IV) may be prepared by treating a compound of formula (V)

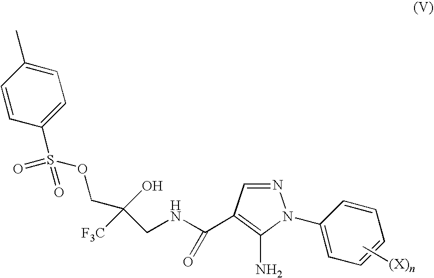

wherein X and n are as defined above for compounds of formula (I),
with a polymer supported carbonate resin. The reaction may be carried out in a conventional organic solvent, for example tetrahydrofuran. The reaction may be carried out at a temperature of from −10° C. to 100° C., for example at room temperature.

A compound of formula (V) may be prepared by treating a compound of formula (VI)

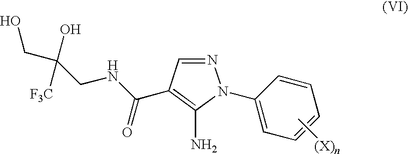

wherein X and n are as defined above for compounds of formula (I),
with 4-methylbenzenesulphonyl chloride. The reaction may be carried out in a conventional organic solvent, for example dichloromethane, in the presence of an organic base, for example pyridine. The reaction may be carried out at a temperature of from −10° C. to 100° C., for example at room temperature.

A compound of formula (VI) may be prepared by reacting a compound of formula (VII)

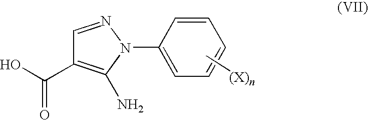

wherein X and n are as defined above for compounds of formula (I), with a compound of formula (VIII)

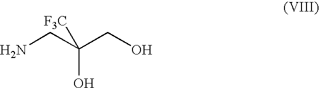

The reaction may be carried out in a conventional organic solvent, for example dimethylformamide, in the presence of a coupling agent such as those described in Tetrahedron 2005, 61, 10827, for example O-(7-azabenzotriazol-1-yl)-N,N,N′,N′-tetramethyluronium hexafluorophosphate (HATU), and a base, for example triethylamine or diisopropylethylamine. In one embodiment, the reaction is carried out in the presence of diisopropylethylamine. The reaction may be carried out at a temperature of from −10° C. to 100° C., for example at room temperature.

Examples of acids of formula (VII) which may be used in this coupling reaction include:
5-amino-1-(2-fluorophenyl)-1H-pyrazole-4-carboxylic acid;
5-amino-1-(3-fluorophenyl)-1H-pyrazole-4-carboxylic acid;
5-amino-1-(4-fluorophenyl)-1H-pyrazole-4-carboxylic acid;
5-amino-1-(4-chlorophenyl)-1H-pyrazole-4-carboxylic acid;
5-amino-1-(3-chlorophenyl)-1H-pyrazole-4-carboxylic acid;
5-amino-1-(2-chlorophenyl)-1H-pyrazole-4-carboxylic acid;
5-amino-1-(2,3-difluorophenyl)-1H-pyrazole-4-carboxylic acid;
5-amino-1-(2,4-difluorophenyl)-1H-pyrazole-4-carboxylic acid;
5-amino-1-(2,5-difluorophenyl)-1H-pyrazole-4-carboxylic acid;
5-amino-1-(2,6-difluorophenyl)-1H-pyrazole-4-carboxylic acid;
5-amino-1-(3,4-difluorophenyl)-1H-pyrazole-4-carboxylic acid;
5-amino-1-(3,5-difluorophenyl)-1H-pyrazole-4-carboxylic acid; and
5-amino-1-phenyl-1H-pyrazole-4-carboxylic acid.

Acids of formula (VII) may be prepared by, for example, reaction of a suitable aryl hydrazine with ethyl 2-cyano-3-ethoxyacrylate followed by conversion of the resulting ethyl ester to the corresponding acid by treatment with, for example, lithium hydroxide in a solvent such as aqueous ethanol.

A compound of formula (VIII) may be prepared by treating a compound of formula (IX)

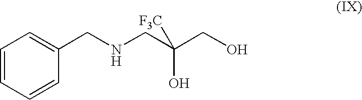

with a transition metal catalyst, for example palladium hydroxide on carbon, in the presence of a hydrogen atmosphere. The reaction may be carried out in a conventional organic solvent, for example ethanol. The reaction may be carried out at a temperature of from −10° C. to 100° C., for example at room temperature.

A compound of formula (IX) may be prepared by treating a compound of formula (X)

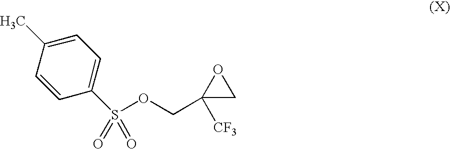

with benzylamine followed by treatment with a base, for example sodium hydroxide. The reaction may be carried out in a conventional organic solvent, for example 1,4-dioxan. The treatment with benzylamine may be carried out at a temperature of from −10° C. to 100° C., for example at room temperature, and the treatment with base may be carried out at a temperature of from −10° C. to 100° C., for example at about 90° C.

A compound of formula (X) may be prepared by treating a compound of formula (XI)

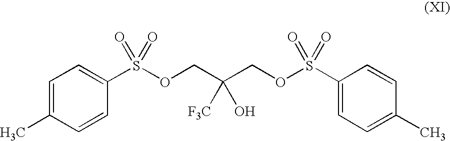

with a polymer supported carbonate resin. The reaction may be carried out in a conventional organic solvent, for example dichloromethane. Batch processes or flow processes are suitable for this cyclo-elimination reaction. The reaction may be carried out at a temperature of from −10° C. to 100° C., for example at room temperature for a batch process or at about 50° C. for a flow process.

A compound of formula (XI) may be prepared by treating a compound of formula (XII)

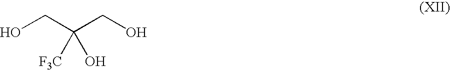

with 4-methylbenzenesulphonyl chloride in the presence of an organic base, for example pyridine. The reaction may be carried out at a temperature of from −10° C. to 100° C., for example at room temperature. Alternatively, when a flow process is used, the compound of formula (XII) may be treated with 4-methylbenzenesulphonyl chloride in the presence of an organic base, for example N,N,N′,N′-tetramethyl-1,6-hexanediamine, in dichloromethane at room temperature. Both batch processes and flow processes are suitable for this reaction.

A compound of formula (XII) may be prepared by treating a compound of formula (XIII)

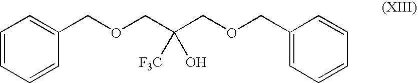

with a transition metal catalyst, for example 5% palladium on carbon, in the presence of a hydrogen atmosphere. The reaction may be carried out in a conventional organic solvent, for example ethanol. The reaction may be carried out at a temperature of from −10° C. to 100° C., for example at room temperature for a batch process or at about 80° C. for a flow process. Batch processes or flow processes are suitable for this hydrogenation.

A compound of formula (XIII) may be prepared by treating a compound of formula (XIV)

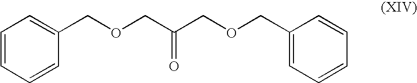

with trimethyl(trifluoromethyl)silane and tetra-n-butylammonium fluoride. The reaction may be carried out in a conventional organic solvent, for example tetrahydrofuran or dichloromethane. The reaction may be carried out at a temperature of from −10° C. to 100° C., for example at 0° C. rising to room temperature. Batch processes or flow processes are suitable for this transformation.

A compound of formula (XIV) may be prepared by oxidation of 1,3-dibenzylglycerol. In one embodiment, the oxidation may be carried out using 3A molecular sieves, N-methylmorpholine N-oxide and tetrapropylammonium perruthenate in dichloromethane at 0° C. to reflux, for example at room temperature. In another embodiment, the oxidation may be carried out using aqueous sodium hypochlorite, saturated sodium bicarbonate solution and 2,2,6,6-tetramethyl-1-piperidinyloxy free radical in toluene at 0° C. to 50° C., for example at room temperature. In a further embodiment, the oxidation may be carried out using sulphur trioxide-pyridine complex in the presence of base such as triethylamine in dimethylsulphoxide at 10° C. to 50° C., for example at room temperature. Batch processes or flow processes are suitable for this oxidation.

Alternatively, compounds of formula (I) may be prepared by coupling a compound of formula (VII) as defined above with a compound of formula (XV),

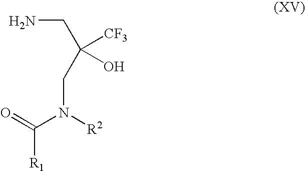

wherein R¹ and R² are as defined above for compounds of formula (I).

The reaction may be carried out using similar conditions to those described above for the reaction of a compound of formula (VII) with a compound of formula (VIII).

A compound of formula (XV) may be prepared by hydrogenating a compound of formula (XVI)

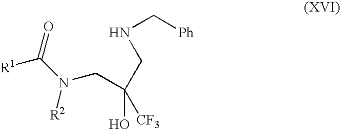

wherein R¹ and R² are as defined above for compounds of formula (I) and Ph is phenyl.

The reaction may be carried out in an organic solvent such as ethanol in the presence of an acid such as 2M hydrochloric acid and a catalyst such as palladium hydroxide on carbon. The reaction may be carried out at a temperature of from 0° C. to 60° C., for example at room temperature.

A compound of formula (XVI) may be prepared by reaction of benzylamine with an epoxide of formula (XVII)

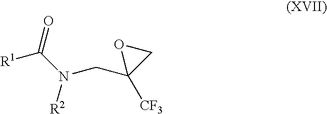

wherein R¹ and R² are as defined above for compounds of formula (I).

The reaction may be carried out in an organic solvent such as tetrahydrofuran at a temperature of from 0° C. to 65° C., for example at room temperature.

A compound of formula (XVII) may be prepared by the reaction of a compound of formula (XVIII)

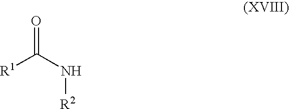

wherein R¹ and R² are as defined above for compounds of formula (I),
with a compound of formula (X) as defined above. The reaction may be carried out in a polar solvent such as tetrahydrofuran, dimethylformamide or dimethoxyethane, preferably dimethoxyethane in the presence of a strong base such as sodium hydride. The reaction may be carried out at a temperature of from −70° C. to +65° C., for example at room temperature.

A compound of formula (XVIII) may be prepared by standard methods from the corresponding amine and acid or acid chloride.

Certain compounds of formulae (II), (III), (IV), (V), (VI), (VIII), (IX), (X), (XI), (XIII), (XV), (XVI), (XVII) and (XVIII) may be new and form an aspect of the present invention.

Compounds of formula (I) may be prepared in the form of mixtures of enantiomers when mixtures of isomers are used as intermediates in the synthesis. For example, the use of a compound of formula (II) as a racemic mixture of enantiomers will lead to a mixture of enantiomers in the final product. These isomers may, if desired, be separated by conventional methods (e.g. HPLC on a chiral column).

Alternatively, separation of isomers may be performed earlier in the synthesis, for example individual isomers of compounds of formula (II) or earlier stage intermediates may be employed which may obviate the need to perform a separation of isomers as a final stage in the synthesis. The later process is, in theory, more efficient and is therefore preferred.

In addition, processes for preparing formulations including one or more compounds of formula (I) form an aspect of this invention.

Compositions comprising a compound of the invention also constitute an aspect of the invention.

Solvates of compounds of formula (I), or salts thereof, which are not physiologically acceptable may be useful as intermediates in the preparation of other compounds of formula (I), salts or solvates thereof.

Compounds of the invention may be expected to demonstrate good anti-inflammatory properties. They also may be expected to have an attractive side-effect profile, demonstrated, for example, by increased selectivity for the glucocorticoid receptor over the progesterone receptor and are expected to be compatible with a convenient regime of treatment in human patients.

The invention will now be illustrated by way of the following non-limiting examples.

### EXAMPLES

The following non-limiting Examples illustrate the invention:

### General

### Abbreviations

| | |
|---|---|
| CO₂ | Carbon dioxide |
| DMF | Dimethylformamide |
| DMSO | Dimethyl sulfoxide |
| E1 | Enantiomer 1 |
| E2 | Enantiomer 2 |
| EtOAc | Ethyl acetate |
| EtOH | Ethanol |
| HATU | O-(7-azabenzotriazol-1-yl)-N,N,N′,N′-tetramethyluronium |
| | hexafluorophosphate |
| HCl | Hydrochloric acid |
| hr | Hours |
| LCMS | Liquid chromatography/mass spectrometry |
| MDAP | Mass directed autopreparative HPLC |
| Me | Methyl |
| MeCN | Acetonitrile |
| MeOD | Deuterated methanol |
| mins | Minutes |
| MgSO₄ | Magnesium sulfate |
| NaCl | Sodium chloride |
| NaHCO₃ | Sodium bicarbonate |
| PEG | Polyethylene glycol |
| Rac | Racemic |
| RT | Room temperature |
| SiO₂ | Silicon dioxide |
| SPE | Solid phase extraction |
| TBAF | Tetrabutyl ammonium fluoride |
| TFA | Trifluoroacetic acid |

### Chromatography

Chromatographic purification was performed using pre-packed Bond Elut silica gel cartridges available commercially from Varian.

The Flashmaster 2 is an automated multi user flash chromatography system which utilises disposable SPE cartridges (2 g to 100 g). It provides quaternary on-line solvent mixing to enable gradient methods to be run. Samples are queued using the multi functional open access software which manages flow rates, gradient profile and collection conditions. The system is equipped with a Knauer variable wavelength uv detector and 2 Gilson FC204 fraction collectors enabling automated peak cutting, collection and tracking.

### Mass Directed Autopreparative HPLC (MDAP)

Agilent 1100 series LC/MSD hardware, using electrospray positive mode (ES+ve) running chemstation 32 purification software.

Column: Zorbax Eclipse XDB-C18 prep HT (dimensions 212×100 mm, 5 μm packing), 20 ml/min solvent speed.

Aqueous solvent=Water+0.1% TFA

Organic solvent=MeCN+0.1% TFA

### Specific Gradients Used:

Gradient 1 (Collects on uv/Mass Ion Trigger)

1 min 70% Water (0.1% TFA): 30% MeCN (0.1% TFA) increasing over 9 mins to 5%
Water (0.1% TFA): 95% MeCN (0.1% TFA) to elute compounds.

### Gradient 2 (Collects on uv Only)

1 min 70% Water (0.1% TFA): 30% MeCN (0.1% TFA) increasing over 9 mins to 5%
Water (0.1% TFA): 95% MeCN (0.1% TFA) to elute compounds.

### CAT MDAP System

Column details: Zorbax Eclipse XDB-C18 prep HT (dimensions 212×100 mm, 5 μm packing)
Cat_norm method, collects on uv/Mass ion trigger
Agilent 1100 series LC/MSD hardware, running chemstation 32 purification software
20 ml/min solvent speed, gradient elution:
1 min 90% Water (0.1% TFA):10% MeCN (0.1% TFA) increasing over 9 mins to 5%
Water (0.1% TFA):95% MeCN (0.1% TFA) to elute compounds.
Cat_gr method, collects on uv/mass ion trigger
1 min 70% Water (0.1% TFA):30% MeCN (0.1% TFA) increasing over 9 mins to 5%
Water (0.1% TFA):95% MeCN (0.1% TFA) to elute compounds.
Cat_lipo uv method is the same as Cat_gr, collecting on uv only

### LCMS System

The LCMS system used was as follows:
Column: 3.3 cm×4.6 mm ID, 3 μm ABZ+PLUS from Supelco
Flow Rate: 3 ml/min
Injection Volume: 5 μl
Temp: RT
UV Detection Range: 215 to 330 nm
   Solvents: A: 0.1% Formic Acid+10 mMolar Ammonium Acetate.
B: 95% Acetonitrile+0.05% Formic Acid

Gradient:

| Time | A % | B % |
|---|---|---|
| 0.00 | 100 | 0 |
| 0.70 | 100 | 0 |
| 4.20 | 0 | 100 |
| 5.30 | 0 | 100 |
| 5.50 | 100 | 0 |

### NMR

¹H NMR spectra were recorded in DMSO-d₆ or chloroform-d or CD₃OD on a Bruker DPX 400, a Bruker AV 400 working at 400 MHz or a Bruker DPX 250 working at 250 MHz. The internal standard used was either tetramethylsilane or the residual protonated solvent at 2.50 ppm for DMSO-d₆ or at 7.27 ppm for chloroform-d₆ or at 3.35 ppm for CD₃OD.

### Circular Dichroism

Circular dichroism was carried out on an Applied Photophysics Chirascan spectrophotometer at room temperature, using acetonitrile as solvent, over the range 200-350 nm.

### Intermediate 1: 1,3-Bis[(benzyl)oxy]-2-propanone

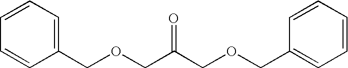

3A Molecular sieve powder (50 g) was dried at 100° C. in a vacuum oven. The sieves and N-methylmorpholine N-oxide (35.1 g, 300 mmol) were suspended in dry dichloromethane (700 ml) before 1,3-dibenzyloxy-2-propanol (41 ml, 165 mmol) in dichloromethane (100 ml) was added to the stirred suspension. The mixture was stirred under an atmosphere of nitrogen for 90 mins before tetrapropylammonium perruthenate (3 g, 8.53 mmol) was added. (The reaction was sufficiently exothermic to cause the dichloromethane to boil and therefore a reflux condenser was fitted.) The reaction was stirred at 21° C. for 23 hr before being filtered through celite. It was then washed with 2M hydrochloric acid (400 ml) and saturated brine (500 ml). The combined aqueous washings were filtered through celite and re-extracted with dichloromethane (500 ml) and then this was washed with saturated brine (200 ml). The organic layers were combined, dried over magnesium sulphate and concentrated under reduced pressure to give a dark oil (43.6 g). Diethyl ether (ca 200 ml) was added and the resultant black solid was filtered off. The filtrate was concentrated under reduced pressure to give the title compound (42 g) as grey white solid.

1H NMR (400 MHz, CHLOROFORM-d) δ ppm 7.31-7.40 (m, 10H) 4.59 (s, 4H) 4.26 (s, 4H).

LC-MS Retention Time 3.27 mins, MNH₄+ 288.

### Alternative Preparation A of Intermediate 1

A mixture of sodium hypochlorite (100 ml, 13% w/v) and saturated sodium bicarbonate (25 ml) was added in one charge to a stirred solution of 1,3-dibenzyloxy-2-propanol (10 g), 2,2,6,6-tetramethyl-1-piperidinyloxy, free radical, (TEMPO) (0.3 g) in toluene (40 ml). The biphasic mixture was stirred at 20-25° C. for 15 mins when HPLC analysis showed reaction to be complete. The reaction mixture was stirred for a total of 25 mins at 23° C. The reaction mixture was separated and the organic extract was washed with 5% w/v sodium thiosulfate solution (40 ml) and separated. The organic extract was washed with 1% w/v sodium chloride solution (2×25 ml). The organic extract was then concentrated in vacuo to give an oil which crystallised on standing to give 8.8 g of 1,3-bis[(benzyl)oxy]-2-propanone in 88.7% th yield. The NMR spectrum of the product was concordant with a reference sample.

### Alternative Preparation B of Intermediate 1

A mixture of sulfur trioxide/pyridine complex (2.33 g, 4 equivalents) and triethylamine (2.05 ml, 4 equivalents) in DMSO (3 ml) was stirred to give a pale yellow solution. To this was added a solution of 1,3-dibenzyloxy-2-propanol (1 g) in DMSO (1 ml) over 2 mins. (The reaction mixture was kept in a water bath). The temperature of the reaction mixture reached 30° C. After 10 mins the water bath was removed and the reaction mixture was stirred at room temperature (ca 20-25° C.) for 3 hr. The reaction mixture was diluted with ethyl acetate (15 ml) and water (15 ml), stirred and the organic extract was separated. The organic extract was washed with 5% w/v sodium chloride (2×10 ml) and water (10 ml). The separated organic extract was concentrated in vacuo to give an oil which solidified to provide 0.75 g of 1,3-bis[(benzyl)oxy]-2-propanone in 75.8% theoretical yield. An NMR spectrum of product was concordant with a reference sample.

### Alternative Preparation C of Intermediate 1

The title compound was prepared via a ‘flow’ process using the following starting materials and solvents.

The title compound was prepared via a CPC Cytos Lab System made up of a 47 ml reactor block with two Jasco PU—2080Plus HPLC pumps. Reactor temperature was maintained at 60° C. via a Huber Unistat 360 unit.

Two solutions were prepared. Solution A—1,3-dibenzyloxy-2-propanol (120 g, 440 mmol) in acetonitrile (489 ml). Solution B—tetrapropylammonium perruthenate (7.72 g, 22 mmol, 5 mol %) and N-methylmorpholine N-oxide (87.5 g, 748 mmol) in acetonitrile (611 ml). Solutions A and B were pumped through the Cytos Lab system in the ratio of solution A to solution B of 1:1.25 with a total flow rate of 7.8 ml/min and residence time of 6 min. This gave a total reaction time of 2 hr 21 mins. The total reacted solution was split equally into 2 batches and each was concentrated in vacuo. Diethyl ether (250 ml) was added before being washed with sodium sulphite, brine, cupric sulphate then filtered through celite, dried and evaporated. The batches were recombined to give upon evaporation in vacuo the title compound (71.64 g).

### Intermediate 2: 1,1,1-Trifluoro-3-[(benzyl)oxy]-2-{[(benzyl)oxy]methyl}-2-propanol

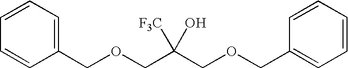

To a solution of 1,3-bis[(benzyl)oxy]-2-propanone (42 g, 155 mmol) in anhydrous tetrahydrofuran (600 ml) was added trimethyl(trifluoromethyl)silane (35 ml, 236 mmol). The mixture was then cooled in an ice/ethanol bath to −3° C. before tetrabutylammonium fluoride (1M in THF, 180 ml, 180 mmol) was added dropwise (initial 10 ml of addition resulted in a slight exotherm with the temperature rising to 9° C. before being allowed to cool to 6° C. and then the addition was resumed, the temperature dropping to the range of −1° C. to +3° C.). The addition was completed after 30 mins. The mixture was stirred for a further 4 hr during which, gas was evolved all the time and then 2M hydrochloric acid (750 ml) was added with stirring. Diethyl ether (600 ml) was added and the separated aqueous phase was reextracted with diethyl ether (1×600 ml, 1×300 ml) and the combined organic extracts were washed with saturated brine (1×300 ml), dried over sodium sulphate and concentrated under reduced pressure to give an oil (52.9 g). This oil was purified via flash chromatography (Silica, 800 g) using cyclohexane:ethyl acetate (9:1) as eluent. This gave the title compound as a yellow oil (39.5 g).

1H NMR (400 MHz, CHLOROFORM-d) δ ppm 7.29-7.39 (m, 10H) 4.60 (s, 4H) 3.72 (s, 4H) 3.38 (s, 1H).

LC-MS Retention Time 3.69 mins, MNH₄+ 358.

### Alternative Preparation A of Intermediate 2

A mixture of 1,3-bis[(benzyl)oxy]-2-propanone (2 g) and (trifluoromethyl)trimethylsilane (2.56 ml, 2.3 equivalents) in dichloromethane (20 ml) was stirred and cooled to 0° C. A solution of 1M tetrabutylammonium fluoride in THF (4 ml) was added dropwise over 3 mins. Initial addition of a few drops gave an exotherm of 10° C. Throughout the addition the batch temperature was maintained below 10° C. After completing the addition the dark brown mixture was stirred at +5° C. for 5 mins when HPLC analysis indicated the reaction to be complete. The reaction mixture was stirred for an additional 5 mins and then washed with 1M aqueous hydrochloric acid (2×15 ml), saturated sodium bicarbonate (15 ml) and 1% w/v aqueous sodium chloride solution (2×15 ml). The organic extract was concentrated in vacuo to give 2.5 g of the desired product as dark oil in 99.3% theoretical yield. The NMR spectrum of the product was concordant with a reference sample.

### Alternative Preparation B of Intermediate 2

Tetrabutylammonium fluoride trihydrate (TBAF 3H₂O) (2.9 g, 0.5 equivalent) was dissolved in THF (5 ml). This was added cautiously to a stirred and cooled (+15° C.) solution of 1,3-bis[(benzyl)oxy]-2-propanone in toluene (24.65 g, equivalent to 5 g of the ketone) and (trifluoromethyl)trimethylsilane (7.5 ml). There was an exotherm and a lot of gas evolution on addition of the first 1 ml of TBAF solution. The temperature rose from 18 to 40° C. The TBAF addition was carried out over 3 mins and then the mixture was stirred at 15-30° C. for a further 2 mins and then cooled to +10° C. while carrying out an HPLC analysis. The reaction mixture was sequentially washed with 1N aqueous hydrochloric acid (50 ml), 1% aqueous sodium chloride solution (2×25 ml) and a mixture of 1% sodium chloride (25 ml) and saturated sodium bicarbonate (5 ml) solution. The separated organic extract was concentrated in vacuo to give 6.41 g of the desired product as dark brown oil in 101.8% th yield. The NMR spectrum showed the presence of residual toluene (8.8%) and starting material (ca 3%).

### Alternative Preparation C of Intermediate 2

The title compound was prepared via a ‘flow’ process using the following starting materials and solvents.

The title compound was prepared via a CPC Cytos Lab System made up of a 32 ml reactor block with two Jasco PU—2080Plus HPLC pumps. Reactor temperature was maintained at 22° C. via a Huber Unistat 360 unit. The reactor outlet was fitted with a 100 psi backflow regulator.

Two solutions were prepared. Solution A—1,3-bis[(benzyl)oxy]-2-propanone (71.64 g, 265 mmol) and trimethyl(trifluoromethyl)silane (86.67 g, 96 ml, 609.5 mmol) in tetrahydrofuran (99 ml). Solution B—tetrabutylammonium fluoride (1M in THF, 265 ml, 132.5 mmol).

Solutions A and B were pumped through the Cytos Lab System with a flow rate of 6.4 ml/min and a 5 min residence time giving a total reaction time of 82 mins. The reaction mixture was quenched with 2M hydrochloric acid (30 ml) and then divided into 2 equal batches. Diethyl ether (100 ml) was added, extracted and then washed with brine (2×100 ml), dried (MgSO₄) and evaporated to give a residue (82.99 g). Part of the residue was taken up in dichloromethane and applied to SPE silica cartridges. Using 10% hexane in dichloromethane as eluent and concentration of the relevant 15 ml fractions, the title compound was obtained. The bulk of the crude sample was purified on the Combiflash Companion XL. 8 g of material was run on a 120 g column with a solvent gradient of 10%-70% dichloromethane in hexane as eluent. Any mixed fractions from each run were combined and repurified in an identical manner. All pure fractions were combined and evaporated to give the title compound (68.68 g).

### Intermediate 3: 2-(Trifluoromethyl)-1,2,3-propanetriol

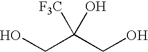

A solution of 1,1,1-trifluoro-3-[(benzyl)oxy]-2-{[(benzyl)oxy]methyl}-2-propanol (98.9 g, 290.9 mmol) in ethanol (1750 ml) was added to 5% palladium on carbon (9.73 g, wet, Degussa, E101 No/W) under nitrogen. The mixture was then stirred under an atmosphere of hydrogen using a Wright valve in a 5 litre hydrogenation vessel. After approximately 3 hr most of the theoretical volume of hydrogen had been taken up (approximately a further 1 litre of hydrogen had been taken up overnight). After stirring under hydrogen overnight, the catalyst was filtered off through a pad of celite and the pad washed with ethanol. The filtrate and washings were then concentrated under reduced pressure and the residue azeotroped (×2) with dichloromethane whereupon the residue became solid. This material was left on the vacuum pump at 40° C. overnight to give the title compound (48.56 g) as an off white solid.

1H NMR (400 MHz, DMSO-d₆) δ ppm 5.65 (s, 1H) 4.89 (t, 2H) 3.54 (d, J=5.8 Hz, 4H).

LC-MS Retention Time 0.42 mins, ES⁻ MH⁻ 159.

### Alternative Preparation A of Intermediate 3

The title compound was prepared employing the Thales H-Cube hydrogenator and milligat pump in full hydrogen mode. A solution of 1,1,1-trifluoro-3-[(benzyl)oxy]-2-{[(benzyl)oxy]methyl}-2-propanol (58 g) in ethanol (580 ml) was prepared. The flow rate was 1.3 ml/min, the temperature was set to 80° C. and the cartridge employed was a 10% Pd/C Cat Cart 70 which was replaced every 2 hr. Any fractions which still contained starting material and the mono benzyl intermediate were reprocessed. All pure fractions were combined and evaporated to give the title compound (26.48 g).

### Intermediate 4: 3,3,3-Trifluoro-2-hydroxy-2-({[(4-methylphenyl) sulfonyl]oxy}methyl)propyl 4-methylbenzenesulfonate

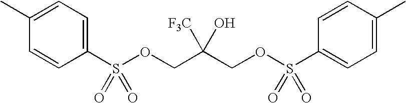

To a stirred solution of 2-(trifluoromethyl)-1,2,3-propanetriol (18.9 g, 118 mmol) in pyridine (200 ml) which had been cooled in an ice bath was added p-toluenesulphonyl chloride (67 g, 351 mmol) to give an orange solution. The ice bath was removed after 45 mins and stirring was continued for 21 hr during which time a solid formed. Most of the pyridine was removed under reduced pressure and the residue was partitioned between ethyl acetate (500 ml) and water (300 ml). The separated aqueous phase was further extracted with ethyl acetate (1×250 ml) and the combined organic extracts were washed with 2M hydrochloric acid (1×200 ml), water (1×200 ml), saturated sodium bicarbonate (1×200 ml), water (1×200 ml) and saturated brine (1×200 ml) before being dried over sodium sulphate and concentrated under reduced pressure to give an oil (72.8 g). This oil was purified on a Flash silica column (800 g) with cyclohexane:ethyl acetate (5:1) to give the title product (49 g, 95%) as an oil which crystallised on standing.

1H NMR (400 MHz, CHLOROFORM-d) δ ppm 7.78 (d, J=8.3 Hz, 4H) 7.38 (d, J=8.3 Hz, 4H) 4.18 (s, 4H) 3.66 (s, 1H) 2.48 (s, 6H).

LC-MS Retention Time 3.62 mins, MNH₄+ 486.

### Alternative Preparation A of Intermediate 4

The title compound was prepared via a flow process using the following starting materials and solvents.

Two solutions were prepared. Solution A—2-(trifluoromethyl)-1,2,3-propanetriol (4.5 gm, 27.8 mmol), N,N,N′,N′-tetramethyl-1,6-hexanediamine (30 ml, 139 mmol), dichloromethane (550 ml). Solution B—p-toluenesulphonyl chloride (21.4 g, 111 mmol), dichloromethane (550 ml).

Solutions A and B were pumped through a CPC Cytos reactor (reactor volume 47 ml) at a flow rate each of 2.35 ml/min. It was noted that the pressure for the pump containing solution B was fluctuating. After 110 mins, the reaction was abandoned as it was evident that the pumps were not operating 1:1. The collected material was extracted with dichloromethane (×3) before being washed with brine, dried (MgSO₄), filtered and concentrated to give a residue which was discarded. The pump was replaced and the remainder of the reagents were reacted. The collected material was extracted with dichloromethane (×3) before being washed with brine, dried (MgSO₄), filtered and concentrated to give a residue. It was adsorbed onto silica and eluted over a silica column (12 g) with dichloromethane:hexane (1:1). Four fractions were eluted and fraction 4 gave the title compound (2.31 g).

### Intermediate 5: [2-(Trifluoromethyl)-2-oxiranyl]methyl 4-methylbenzenesulfonate

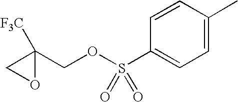

A solution of the bis tosylate, 3,3,3-trifluoro-2-hydroxy-2-({[(4-methylphenyl) sulfonyl]oxy}methyl)propyl 4-methylbenzenesulfonate (186.5 g, 398.5 mmol) in dichloromethane (2500 ml) was stirred under nitrogen whilst polymer supported carbonate resin (ex Fluka, ca. 3.5 mmoles carbonate/g resin) (232 g) was added. The mixture was stirred at room temperature overnight. The resin was filtered off and the resin was washed with dichloromethane. The combined filtrate and washings were concentrated under reduced pressure to give the title compound (116.2 g) as a brown oil.

1H NMR (400 MHz, CHLOROFORM-d) δ ppm 7.80 (d, J=8.3 Hz, 2H) 7.38 (d, J=8.0 Hz, 2H) 4.41 (d, J=11.9 Hz, 1H) 4.29 (d, J=11.9 Hz, 1H) 3.14 (d, J=4.8 Hz, 1H) 3.01 (dd, J=4.5, 1.5 Hz, 1H) 2.47 (s, 3H).

LC-MS Retention Time 3.2 mins, MNH₄+ 314.

### Alternative Preparation A of Intermediate 5

3,3,3-Trifluoro-2-hydroxy-2-({[(4-methylphenyl)sulfonyl]oxy}methyl)propyl 4-methylbenzenesulfonate (14.29 g, 29 mmol) in dichloromethane (75 ml) was pumped through a cartridge containing PS-carbonate resin (not pre-swelled) (3 mmol/g, 25 g, 75 mmol) at 675 microlitres/min. The temperature was set to approx. 50° C. by wrapping a Whatman thin film heater around the cartridge. The pressure was regulated at 40 psi. After all the reagent had been aspirated, the column was washed through with dichloromethane—at this point the column started to leak slightly and the BPR had to be removed to reduce the pressure. The collected solution was concentrated in vacuo to afford the title compound (7.24 g).

### Intermediate 6: 3,3,3-Trifluoro-2-{[(phenylmethyl)amino]methyl}-1,2-propanediol

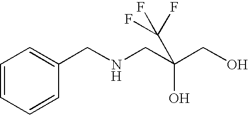

To a stirred solution of the [2-(trifluoromethyl)-2-oxiranyl]methyl 4-methylbenzenesulfonate (10.07 g, 34 mmol) in anhydrous 1,4-dioxan (70 ml) cooled in an ice bath was added benzylamine (4.1 ml, 37.4 mmol) in small portions over 10 mins. The mixture was stirred at ice bath temperature for a further hour before being allowed to warm to 21° C. and then stirred for 18 hr. 2M Sodium hydroxide (50 ml) and 1,4-dioxan (50 ml) were added and stirred for 2 hr at room temperature before being heated at 90° C. for 22 hr. The mixture was allowed to cool before being concentrated to low volume and partitioned between ethyl acetate (250 ml) and water (100 ml). The separated aqueous layer was further extracted with ethyl acetate (1×250 ml) and the combined organic extracts were washed with water (1×100 ml), saturated brine (1×100 ml), dried over sodium sulphate and concentrated under reduced pressure to give an oil (9.4 g). This was purified on 3×10 g SPE cartridges using a 0-100% cyclohexane-ethyl acetate gradient over 60 mins. This gave upon concentration of the relevant fractions under reduced pressure, the title compound (5.09 g) as an oil.

1H NMR (400 MHz, CHLOROFORM-d) δ ppm 7.30-7.35 (m, 2H) 7.23-7.29 (m, 3H) 3.75-3.86 (m, 4H) 3.57 (d, J=11.6 Hz, 1H) 3.06 (d, J=13.1 Hz, 1H) 2.86 (d, J=13.1 Hz, 1H), OH's & NH are very broad 2.5-3.0 ppm.

LC-MS Retention Time 1.45 mins, MH⁺ 250.

### Intermediate 7: 2-(Aminomethyl)-3,3,3-trifluoro-1,2-propanediol

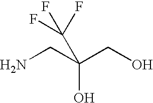

A solution of 3,3,3-trifluoro-2-{[(phenylmethyl)amino]methyl}-1,2-propanediol (8.33 g, 33.4 mmol) in ethanol (550 ml) containing palladium hydroxide on carbon (20%, 800 mg) was stirred under an atmosphere of hydrogen for 24 hr. The catalyst was filtered off via a pad of celite and the filtrate was concentrated under reduced pressure, toluene was added and the solution evaporated again under reduced pressure to give the title compound (5.06 g) as an oil.

1H NMR (400 MHz, MeOD) δ ppm 3.69-3.69 (m, 2H) 2.96 (d, J=13.5 Hz, 1H) 2.87 (d, J=13.5 Hz, 2H).

LC-MS Retention Time 0.32 mins, MH⁺ 160.

### Intermediate 8: Ethyl 5-amino-1-(4-fluorophenyl)-1H-pyrazole-4-carboxylate

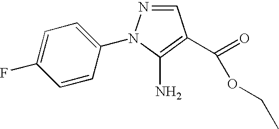

To a stirred suspension of 4-fluorophenylhydrazine hydrochloride (9.76 g, 60 mmol) in ethanol (250 ml) was added triethylamine (9.2 ml, 62 mmol) and to the resulting amber solution was added ethyl 2-cyano-3-ethoxyacrylate (10.15 g, 60 mmol). The solution was heated at reflux temperature for 3.5 hr. The solution was allowed to cool to room temperature and after standing overnight the resultant solid was filtered off, washed with small amount of ethanol and then ether before being dried under vacuum to give the title compound (12.1 g) as an off white solid.

1H NMR (400 MHz, DMSO-d₆) δ ppm 7.70 (s, 1H) 7.55 (s, J=5.0 Hz, 2H) 7.34-7.41 (m, 2H) 6.34 (br. s., 2H) 4.21 (q, J=7.0, 7.0 Hz, 2H) 1.26 (t, J=7.0 Hz, 3H).

### Intermediate 9: 5-Amino-1-(4-fluorophenyl)-1H-pyrazole-4-carboxylic acid

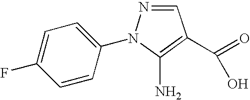

To a suspension of ethyl 5-amino-1-(4-fluorophenyl)-1H-pyrazole-4-carboxylate (12.1 g, 48.5 mmol) in ethanol (250 ml) was added a solution of lithium hydroxide (5.8 g, 242 mmol) in water (100 ml). The mixture was stirred at reflux for 2.5 hr. It was allowed to cool and concentrated to 50% of its volume before 5M hydrochloric acid (47 ml) was added. After stirring for 15 mins, the resulting white solid was filtered off and further 5M hydrochloric acid (3 ml) was added. This was filtered and the combined solids were washed with water and diethyl ether and then dried under vacuum to give the title compound (10.27 g).

1H NMR (400 MHz, DMSO-d₆) δ ppm 12.09 (br. s., 1H) 7.67 (s, 1H) 7.54-7.60 (m, 2H) 7.34-7.41 (m, 2H) 6.29 (br. s., 2H).

LC-MS Retention Time 2.20 mins, MH⁺ 222.

### Intermediate 10: 5-Amino-1-(4-fluorophenyl)-N-[3,33-trifluoro-2-hydroxy-2-(hydroxymethyl)propyl]-1H-pyrazole-4-carboxamide

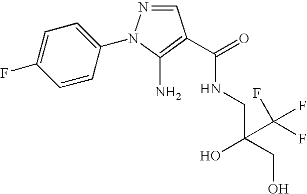

A solution of 5-amino-1-(4-fluorophenyl)-1H-pyrazole-4-carboxylic acid (5.86 g, 26.5 mmol) in anhydrous dimethylformamide (60 ml) and diisopropylethylamine (17.5 ml, 100 mmol) was cooled in an ice bath for 5 mins before O-(7-azabenzotriazol-1-yl)-N,N,N′,N′-tetramethyluronium hexafluorophosphate (HATU) (11.1 g, 29.2 mmol) was added. After 5 mins, the solution was removed from the ice bath and stirred under nitrogen for 20 mins. The stirred mixture was recooled in ice for 5 mins before a solution of the amine, 2-(aminomethyl)-3,3,3-trifluoro-1,2-propanediol (5 g, 31.4 mmol) in anhydrous dimethylformamide (20 ml) was added. The ice bath was again removed and stirring continued for 2.5 hr. The mixture was then partitioned between ethyl acetate (500 ml) and water (500 ml) and the separated aqueous phase was reextracted with ethyl acetate (300 ml). The combined organic extracts were washed with water (1×500 ml, 1×300 ml), 1M hydrochloric acid (1×400 ml), aqueous lithium chloride (2×200 ml), saturated sodium hydrogen carbonate (1×200 ml), water (200 ml) and saturated brine (2×200 ml) before being dried over sodium sulphate and concentrated under vacuum to give a foam (9.5 g). Ethyl acetate (5 ml) was added followed by dichloromethane (50 ml) and the mixture was swirled to initiate crystallisation. It was left to stand in the fridge for 15 hr and the resultant solid was filtered off and washed with small amounts of dichloromethane and heptane before being dried under vacuum to give the title compound (7.0 g, 73%) as a white solid.

1H NMR (400 MHz, DMSO-d₆) δ ppm 8.17 (t, J=6.1 Hz, 1H) 7.99 (s, 1H) 7.54-7.60 (m, 2H) 7.33-7.40 (m, 2H) 6.37 (br. s., 2H) 6.30 (s, 1H) 5.19 (t, J=6.4 Hz, 1H) 3.51-3.69 (m, 2H) 3.38-3.50 (m, 2H).

LC-MS Retention Time 2.20 mins, MH⁺ 363.

### Intermediate 11: 2-[({[5-Amino-1-(4-fluorophenyl)-1H-pyrazol-4-yl]carbonyl}amino)methyl]-3,3,3-trifluoro-2-hydroxypropyl 4-methylbenzenesulfonate

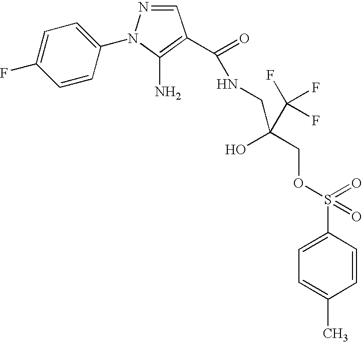

To a stirred solution of 5-amino-1-(4-fluorophenyl)-N-[3,3,3-trifluoro-2-hydroxy-2-(hydroxymethyl)propyl]-1H-pyrazole-4-carboxamide (2.47 g, 6.82 mmol) in anhydrous dichloromethane (20 ml) and anhydrous pyridine (20 ml) cooled in an ice bath under a nitrogen atmosphere was added p-toluenesulphonyl chloride (1.7 g, 8.9 mmol). The mixture was stirred for 6 hr at ice bath temperature before being allowed to warm to room temperature and stirred overnight. The solution was evaporated under vacuum and the residue was partitioned between ethyl acetate (100 ml) and water (30 ml). The separated organic phase was washed with 2M hydrochloric acid (2×30 ml), water (30 ml), saturated sodium hydrogen carbonate (30 ml), water (30 ml) and saturated brine (50 ml) before being dried over sodium sulphate and evaporated under reduced pressure to give a foam (3.45 g). This foam was purified on a Flashmaster column of Silica (100 g) using a 0-100% ethyl acetate in cyclohexane gradient over 1 hr. This afforded the title compound (2.8 g, 79%) as a foam.

1H NMR (400 MHz, DMSO-d₆) δ ppm 8.04 (t, J=6.3 Hz, 1H) 7.90 (s, 1H) 7.78 (d, J=8.1 Hz, 2H) 7.55-7.60 (m, 2H) 7.45 (d, J=8.1 Hz, 2H) 7.34-7.41 (m, 2H) 3.97-4.06 (m, 2H) 3.63 (dd, J=14.7, 6.6 Hz, 1H) 3.45 (dd, 1H) 2.38 (s, 3H).

LC-MS Retention Time 3.41 mins, MH⁺ 517.

### Intermediate 12: 5-Amino-1-(4-fluorophenyl)-N-{[2-(trifluoromethyl)-2-oxiranyl]methyl}-1H-pyrazole-4-carboxamide

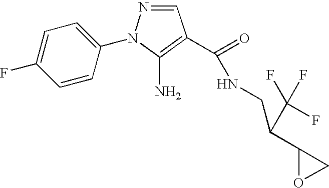

A solution 2-[({[5-amino-1-(4-fluorophenyl)-1H-pyrazol-4-yl]carbonyl}amino)methyl]-3,3,3-trifluoro-2-hydroxypropyl 4-methylbenzenesulfonate (2.8 g, 5.4 mmol) in anhydrous tetrahydrofuran (150 ml) was shaken with polymer supported carbonate resin (ex Fluka, 5 g, 3.5 mmol/g, 17.5 mmol) which had been prewashed with tetrahydrofuran (5×). After shaking for 15 hr, the resin was filtered off and the filtrate was evaporated under reduced pressure to give a semi-solid (1.986 g). Diethyl ether (ca 10 ml) was added and after standing for 3 hr, the resultant crystallised solid (1 g) was filtered off and washed with heptane. The filtrate and the solid which had precipitated were evaporated under reduced pressure and the residue, dissolved in dichloromethane, was purified on a Flashmaster silica column (100 g) eluting with 0-100% ethyl acetate in cyclohexane over 60 mins to afford a cream solid (0.46 g). This was combined with the crystallised solid to give the title compound (1.46 g).

1H NMR (400 MHz, DMSO-d₆) δ ppm 8.15 (t, J=6.1 Hz, 1H) 7.94 (s, 1H) 7.53-7.60 (m, 2H) 7.36 (t, J=8.8 Hz, 2H) 6.38 (s, 2H) 3.88 (dd, J=14.9, 6.1 Hz, 1H) 3.69 (dd, J=14.8, 6.0 Hz, 1H) 3.17 (d, J=4.3 Hz, 1H) 2.90-2.98 (m, J=4.0 Hz, 1H).

LC-MS Retention Time 2.83 mins, MH⁺ 345.

### Intermediate 13: 5-Amino-N-{2-[(ethylamino)methyl]-3,3,3-trifluoro-2-hydroxypropyl}-1-(4-fluorophenyl)-1H-pyrazole-4-carboxamide

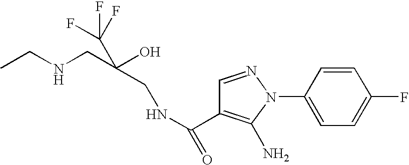

To a solution of 5-amino-1-(4-fluorophenyl)-N-{[2-(trifluoromethyl)-2-oxiranyl]methyl}-1H-pyrazole-4-carboxamide (2 g, 5.8 mmol) in acetonitrile (25 ml) was added ethylamine (5 ml, 23 mmol). The solution was stirred at room temperature for 24 hr under nitrogen before further ethylamine (2 ml) was added. The solution was concentrated under reduced pressure to give the title compound (2.289 g).

1H NMR (400 MHz, CHLOROFORM-d) δ ppm 7.64 (s, 1H) 7.50-7.56 (m, 2H) 7.19-7.25 (m, 2H) 6.29 (br. s., 1H) 5.46 (s, 2H) 3.83 (dd, J=14.4, 7.6 Hz, 1H) 3.60 (dd, J=14.3, 4.7 Hz, 1H) 3.08 (d, J=13.4 Hz, 1H) 2.63-2.84 (m, 3H) 1.13 (t, 3H).

LC-MS Retention Time 2.06 mins, MH⁺ 390.

### Alternative Preparation A of Intermediate 13

A solution of 5-amino-N-(2-{[ethyl(phenylmethyl)amino]methyl}-3,3,3-trifluoro-2-hydroxypropyl)-1-(4-fluorophenyl)-1H-pyrazole-4-carboxamide (0.39 g, 0.813 mmol) in ethanol (35 ml) was stirred under an atmosphere of hydrogen over Pearlman's catalyst (45 mg) for 7 hr (32 ml of hydrogen taken up). The catalyst was filtered off and the filtrate was evaporated to give the title compound (0.313 g) as a white solid.

1H NMR (400 MHz, DMSO-d₆) δ ppm 8.10 (t, 1H) 7.94 (s, 1H) 7.54-7.61 (m, 2H) 7.33-7.40 (m, 2H) 6.37 (br. s., 2H) 3.65 (dd, J=6.3 Hz, 1H) 3.46 (dd, J=14.0, 5.7 Hz, 1H) 2.72 (dd, J=5.6 Hz, 2H) 2.52-2.62 (m, 2H) 1.01 (t, J=7.1 Hz, 3H).

LC-MS Retention Time 2.00 mins, MH⁺ 390.

### Intermediate 14: 5-Amino-1-(4-fluorophenyl)-N-(3,3,3-trifluoro-2-{[(2-fluoroethyl)amino]methyl}-2-hydroxypropyl)-1H-pyrazole-4-carboxamide

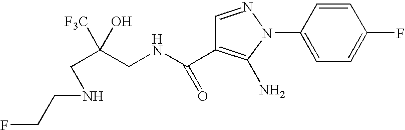

To a solution of 5-amino-1-(4-fluorophenyl)-N-{[2-(trifluoromethyl)-2-oxiranyl]methyl}-1H-pyrazole-4-carboxamide (1.03 g, 3 mmol) in anhydrous acetonitrile (10 ml) was added 2-fluoroethylamine hydrochloride (650 mg, 6 mmol, ca 90% purity) and triethylamine (0.98 ml, 7 mmol) and the resulting suspension was shaken for 4 days. It was partitioned between ethyl acetate (70 ml) and water (20 ml), washed with saturated brine (20 ml), dried over sodium sulphate and evaporated to give a gum (1.57 g). This was purified on 100 g SiO₂ using the Flashmaster 2 with 0-100% ethyl acetate in cyclohexane 60 mins gradient as eluent. This gave the title compound (924 mg).

1H NMR (400 MHz, DMSO-d₆) δ ppm 7.98-8.08 (m, 1H) 7.94 (s, 1H) 7.53-7.61 (m, 2H) 7.32-7.40 (m, 2H) 6.37 (s, 2H) 4.53 (t, 1H) 4.41 (t, 1H) 3.61-3.72 (m, 1H) 3.43-3.53 (m, 1H) 2.74-2.92 (m, 4H)

LC-MS Retention Time 2.12 mins. MH⁺ 408.

### Intermediate 15: 3-Amino-2-{[ethyl(phenylmethyl)amino]methyl}-1,1,1-trifluoro-2-propanol

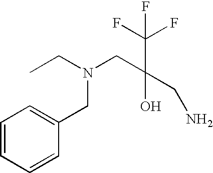

To a solution of [2-(trifluoromethyl)-2-oxiranyl]methyl 4-methylbenzenesulfonate (4.15 g, 14 mmol) in anhydrous dioxan (35 ml) was added N-ethyl benzylamine (2.4 ml, 16.1 mmol). The mixture was stirred under nitrogen at 21° C. for 24 hr. 0.5M Ammonia in dioxan (200 ml, 100 mmol) was added, stirred for 30 min and then heated at 100° C. for 24 hr. It was allowed to cool, the solid filtered off and the filtrate evaporated under reduced pressure to give a residue (4.7 g). This was purified on Flashmaster (3×100 g silica cartridges) using a gradient of 0-25% methanol in dichloromethane over 60 mins to give the title compound (2.64 g) as an oil.

1H NMR (400 MHz, DMSO-d₆) δ ppm 7.17-7.38 (m, 5H) 3.84 (d, J=13.8 Hz, 1H) 3.56 (d, J=13.8 Hz, 1H) 2.79 (s, 2H) 2.62-2.76 (m, 2H) 2.53-2.61 (m, 1H) 2.38-2.48 (m, J=13.4, 6.9, 6.8 Hz, 1H) 0.94 (t, J=7.0 Hz, 3H).

LC-MS Retention Time 2.18 mins, MH⁺ 277.

### Intermediate 16: 5-Amino-N-(2-{[ethyl(phenylmethyl)amino]methyl}-3,3,3-trifluoro-2-hydroxypropyl)-1-(4-fluorophenyl)-1H-pyrazole-4-carboxamide

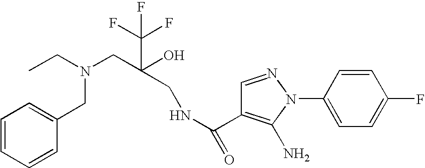

Diisopropylethylamine (0.175 ml, 1 mmol) was added to a mixture of 5-amino-1-(4-fluorophenyl)-1H-pyrazole-4-carboxylic acid (0.119 g, 0.54 mmol) and O-(7-azabenzotriazol-1-yl)-N,N,N′,N′-tetramethyluronium hexafluorophosphate (HATU) (0.205 g, 0.54 mmol) in dimethylformamide (1 ml). The resultant mixture was stirred at room temperature for 20 mins, then a solution of 3-amino-2-{[ethyl(phenylmethyl)amino]methyl}-1,1,1-trifluoro-2-propanol (0.296 g, 1.07 mmol) in dimethylformamide (1 ml) was added. The reaction mixture was stirred at room temperature for 4 hr. The reaction mixture was partitioned between ethyl acetate (30 ml) and water (30 ml) and the separated organic phase washed with saturated aqueous sodium chloride solution, dried over magnesium sulphate, filtered and evaporated under reduced pressure. The residue was purified by chromatography on silica (10 g) using 0-100% ethyl acetate in cyclohexane gradient over 15 mins to give the title compound (0.39 g).

1H NMR (400 MHz, CHLOROFORM-d) δ ppm 7.50-7.55 (m, 2H) 7.49 (s, 1H) 7.25-7.36 (m, 5H) 7.19-7.24 (m, 2H) 5.83-5.89 (m, 1H) 5.71 (br. s., 1H) 5.46 (br. s., 2H) 3.78-3.87 (m, 2H) 3.63 (d, J=13.4 Hz, 1H) 3.48 (dd, J=14.1, 4.5 Hz, 1H) 2.98 (d, J=14.9 Hz, 1H) 2.77 (d, J=15.2 Hz, 1H) 2.56-2.72 (m, 2H) 1.06 (t, J=7.1 Hz, 3H).

LC-MS Retention Time 2.58 mins, MH⁺ 480.

### Intermediate 17: N-Ethyl-N-(3,3,3-trifluoro-2-hydroxy-2-{[(phenylmethyl)amino]methyl}propyl)benzamide

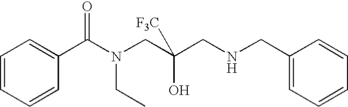

To a solution of N-ethylbenzamide hydrochloride (0.894 g, 6 mmol) in DMF (2.5 ml) was added sodium hydride (0.160 g, 6.6 mmol). After 20 mins of stirring [2-(trifluoromethyl)-2-oxiranyl]methyl 4-methylbenzenesulfonate (1.776 g, 6 mmol) was added and stirring continued overnight. After 2 hr benzylamine (8.27 ml) was added and stirring continued. After 24 hr the reaction mixture was dissolved in EtOAc (50 ml) then washed with water (50 ml), NaHCO₃ (40 ml), sodium chloride (2×20 ml), water (20 ml) and brine (20 ml), then dried over MgSO₄ and concentrated to give a yellow oil. The oil was purified by chromatography on silica (100 g) using 0-100% ethyl acetate in cyclohexane gradient over 40 mins to give the title compound (730 mg).

LC-MS Retention Time 2.16 mins, MH⁺ 381.

### Intermediate 18: N-[2-(Aminomethyl)-3,3,3-trifluoro-2-hydroxypropyl]-N-ethylbenzamide

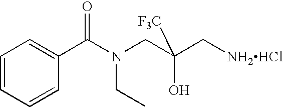

N-Ethyl-N-(3,3,3-trifluoro-2-hydroxy-2-{[(phenylmethyl)amino]methyl}propyl)benzamide (730 mg, 1.92 mmol) was dissolved in EtOH (10 ml) and 4M HCl/dioxane (2 ml) was added, this was then evaporated under vacuum. The solid produced was then dissolved in EtOH and put on the H cube at 50 bar, 25° C. to hydrogenate. LCMS showed that only some product was present so the substrate was put back through the H cube at 60 bar, 40° C. The solution was evaporated under vacuum for 10 mins after which ether (10 ml) was added to make a white solid (536 mg).

LC-MS Retention Time 1.71 mins, MH⁺ 291.

### Example 1

### 5-Amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide

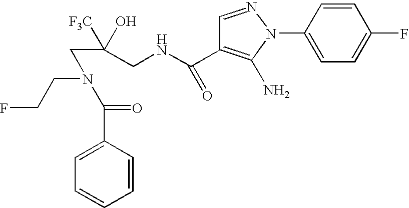

To a solution of 5-amino-1-(4-fluorophenyl)-N-(3,3,3-trifluoro-2-{[(2-fluoroethyl)amino]methyl}-2-hydroxypropyl)-1H-pyrazole-4-carboxamide (160 mg, 0.39 mmol) in anhydrous dichloromethane (5 ml) was added diisopropylethylamine (174 μl, μmol) followed by benzoyl chloride (51 μl, 0.44 mmol). It was left at 21° C. for 18 hr before being washed with 2M hydrochloric acid (2 ml) and water (3 ml) and blown down to give a yellow solid. Dichloromethane (ca. 1 ml) was added to give a white solid which was filtered off and washed with a little dichloromethane and then ether to give the title compound (140 mg).

¹H NMR (400 MHz, DMSO-d₆) δ ppm 7.99-8.09 (m, 1H) 7.89 (s, 1H) 7.52-7.62 (m, 2H) 7.43-7.50 (m, 3H) 7.33-7.43 (m, 4H) 6.33-6.43 (m, 2H) 4.51-4.61 (m, 1H) 4.40-4.49 (m, 1H) 4.02-4.13 (m, 1H) 3.79-3.86 (m, 2H) 3.44-3.55 (m, 1H) 3.30-3.38 (m, 1H).

LC-MS Retention Time 3.12 mins. MH⁺ 512.

A sample of Example 1 was further separated into its enantiomers (Enantiomers 1 and 2) using a 5 cm×20 cm Chiralcel OD column eluting with 70% ethanol in heptane at a flow rate of 75 ml/min.

### Enantiomer 1

Analytical Chiral HPLC (25 cm Chiralcel OD column, 60% ethanol in heptane eluting at 1 ml/min)—Retention time 5.02 mins.

Circular Dichroism (MeCN, RT, 0.000146M, v=350-190 nm, cell length=0.2 cm)

201.4 nm (de=7.41).

264.0 nm (de=3.27).

### Enantiomer 2

Analytical Chiral HPLC (25 cm Chiralcel OD column, 60% ethanol in heptane eluting at 1 ml/min)—Retention time 11.77 mins.

Circular Dichroism (MeCN, RT, 0.000127M, v=350-190 nm, cell length=0.2 cm)
201.4 nm (de=−6.66).
262.0 nm (de=−3.73).

### Example 2

### 5-Amino-1-(4-fluorophenyl)-N-[3,3,3-trifluoro-2-([(2-fluoroethyl)[(2-fluorophenyl)carbonyl]amino]methyl)-2-hydroxypropyl]-1H-pyrazole-4-carboxamide

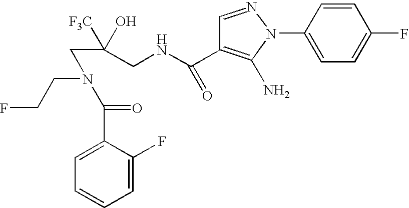

5-Amino-1-(4-fluorophenyl)-N-(3,3,3-trifluoro-2-{[(2-fluoroethyl)amino]methyl}-2-hydroxypropyl)-1H-pyrazole-4-carboxamide (41 mg, 0.1 mmol) was added to a solution of 2-fluorobenzoyl chloride (17 mg, 0.11 mmol) in dichloromethane (400 μl). Diisopropylethylamine (40 μl, 0.3 mmol) was added and the solution was shaken for 5 mins. It was then left to stand at room temperature overnight. The solvent was removed on the Genevac. The residue was purified via CAT MDAP using the Cat_lipo uv method. The solvents were removed on the Genevac to give the title compound (18.8 mg).

1H NMR (600 MHz, DMSO-d₆) δ ppm 8.04 (s, 1H) 7.88 (t, 1H) 7.55-7.61 (m, 2H) 7.48-7.55 (m, 1H) 7.27-7.43 (m, 5H) 4.35-4.77 (m, 1H) 3.65-3.91 (m, 6H)

LC-MS Retention Time 3.13 mins, MH⁺ 512.

A sample of Example 2 was further separated into its enantiomers (Enantiomers 1 and 2) using a 5 cm×20 cm Chiralcel OD column eluting with 60% ethanol in heptane at a flow rate of 15 ml/min.

### Enantiomer 1

Analytical Chiral HPLC (25 cm Chiralcel OD column eluting with 60% ethanol in heptane at a flow rate of 1 ml/min.)—Retention time 4.4 mins.

### Enantiomer 2

Analytical Chiral HPLC (25 cm Chiralcel OD column eluting with 60% ethanol in heptane at a flow rate of 1 ml/min.)—Retention time 9 mins.

### Example 3

### N-[2-[({[5-Amino-1-(4-fluorophenyl)-1H-pyrazol-4-yl]carbonyl]amino)methyl]-3,3,3-trifluoro-2-hydroxypropyl}-3-chloro-N-ethyl-2-Pyridinecarboxamide

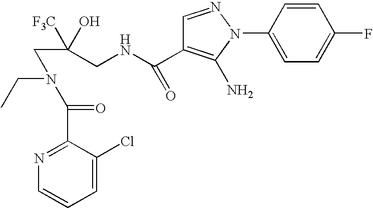

To a solution of O-(7-azabenzotriazol-1-yl)-N,N,N′,N′-tetramethyluronium hexafluorophosphate (HATU) (316 mg, 0.832 mmol) in dimethylformamide was added 3-chloropyridine-2-carboxylic acid (131.1 mg, 0.832 mmol). After ca. 10 mins diisopropylethylamine (292.6 mg, 2.27 mmol) and 5-amino-N-{2-[(ethylamino)methyl]-3,3,3-trifluoro-2-hydroxypropyl}-1-(4-fluorophenyl)-1H-pyrazole-4-carboxamide (294 mg, 0.756 mmol) in dimethylformamide were added. The solution was stirred under nitrogen at room temperature. After 2.5 hr the reaction mixture was concentrated in vacuo. The reaction mixture was partitioned between dichloromethane and water, the organic phase was separated via the use of a hydrophobic frit. The organic layer was then concentrated in vacuo. Purification on silica (50 g) using the Flashmaster using a 0-25% methanol:dichloromethane gradient over 40 mins gave, upon combination and concentration of the appropriate fractions, impure product (700 mg). This was partitioned between ether and water and the organic layer was concentrated in vacuo. Further purification was on silica (50 g) using the Flashmaster 2 eluting with 0-100% ethyl acetate:cyclohexane gradient over 30 mins. Combination and concentration in vacuo of the appropriate fractions gave the title compound (152 mg).

1H NMR (600 MHz, DMSO-d₆) δ ppm 8.61 (d, 1H) 8.12 (d, 1H) 8.04 (t, 1H) 7.85 (s, 1H) 7.55-7.61 (m, 3H) 7.33-7.39 (m, 2H) 6.35 (br. s., 2H) 4.16-4.23 (m, 1H) 3.92-3.98 (m, 1H) 3.47-3.55 (m, 3H) 3.18-3.26 (m, 1H) 1.02-1.08 (m, 3H).

LC-MS Retention Time 3.07 mins, MH⁺ 529.

A sample of Example 3 was further separated into its enantiomers (Enantiomer 1 and 2) using a Chiralpak AD column eluting with 60% isopropanol in heptane at a flow rate of 15 ml/min.

### Enantiomer 1

Analytical Chiral HPLC (25 cm Chiralpak AD column, 60% ethanol in heptane eluting at 1 ml/min)—Retention time 5.0 mins.

Circular Dichroism (MeCN, RT, 0.000140M, v=350-200 nm, cell length=0.2 cm)
215.2 nm (de=−3.79).
271.0 nm (de=−5.09).

### Enantiomer 2

Analytical Chiral HPLC (25 cm Chiralpak AD column, 60% ethanol in heptane eluting at 1 ml/min)—Retention time 8.3 mins.

Circular Dichroism (MeCN, RT, 0.000149M, v=350-200 nm, cell length=0.2 cm)
214.0 nm (de=3.82).
271.0 nm (de=4.97).

### Example 4

### 5-Amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide

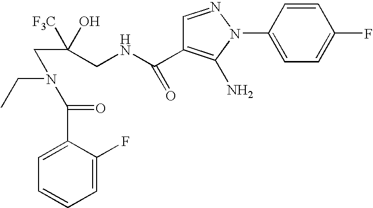

To a solution of 5-amino-N-{2-[(ethylamino)methyl]-3,3,3-trifluoro-2-hydroxypropyl}-1-(4-fluorophenyl)-1H-pyrazole-4-carboxamide (0.1 mmol) in anhydrous dichloromethane (0.5 ml) and anhydrous pyridine (0.5 ml) was added 2-fluorobenzoyl chloride (23.8 μl, 0.2 mmol). After 24 hr, the mixture was diluted with dichloromethane (5 ml), washed with 2M hydrochloric acid (3×3 ml), water (1×3 ml) and saturated brine (5 ml) and blown down. The title product was isolated by MDAP to give (20.5 mg).

1H NMR (400 MHz, MeOD) δ ppm 7.81-7.91 (m, 1H) 7.49-7.59 (m, 4H) 7.38-7.47 (m, 1H) 7.22-7.33 (m, 5H) 4.20-4.39 (m, 1H) 3.96-4.10 (m, 1H) 3.56-3.68 (m, 2H) 3.39-3.51 (m, 2H) 1.01-1.12 (m, 3H).

LC-MS Retention Time 3.2 mins, MH⁺ 512.

A sample of Example 4 was further separated into its enantiomers (Enantiomer 1 and 2) using a 5 cm×20 cm Chiralcel OD column eluting with 20% ethanol in heptane at a flow rate of 75 ml/min.

### Enantiomer 1

Analytical Chiral HPLC (25 cm Chiralcel OD column, 20% ethanol in heptane eluting at 1 ml/min)—Retention time 13.1 mins.

### Enantiomer 2

Analytical Chiral HPLC (25 cm Chiralcel OD column, 20% ethanol in heptane eluting at 1 ml/min)—Retention time 17.2 mins.

### Example 5

### 5-Amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide

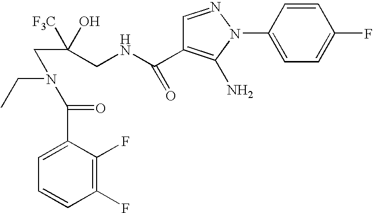

A solution of 2,3-difluorobenzoic acid (0.088 mmol) and O-(7-azabenzotriazol-1-yl)-N,N,N′,N′-tetramethyluronium hexafluorophosphate (HATU) (0.088 mmol) in dimethylformamide (0.2 ml) and N,N-diisopropylethylamine (30 μl) was shaken for 1 min before a solution of 5-amino-N-{2-[(ethylamino)methyl]-3,3,3-trifluoro-2-hydroxypropyl}-1-(4-fluorophenyl)-1H-pyrazole-4-carboxamide (0.07 mmol) in dimethylformamide (0.1 ml) was added and then shaken for a further minute. After standing at room temperature for 18 hrs, dimethylsulphoxide (0.3 ml) was added and this solution was purified via the CAT MDAP (2 times, by cat_gr method and then by cat-lipo uv method) to give the title compound (10.5 mg).

1H NMR (400 MHz, CHLOROFORM-d) δ ppm 7.71-7.75 (m, 1H) 7.49-7.55 (m, 2H) 7.25-7.30 (m, 2H) 7.17-7.25 (m, 4H) 7.06-7.11 (m, 1H) 5.47 (br. s., 2H) 3.98-4.10 (m, 2H) 3.62-3.72 (m, 1H) 3.51-3.57 (m, 1H) 3.43-3.49 (m, 1H) 3.25-3.36 (m, 1H) 1.07 (t, 3H)

LC-MS Retention Time 3.25 mins, MH⁺ 530.

A sample of Example 5 was further separated into its enantiomers (Enantiomer 1 and 2) using a Chiralpak AD column eluting with 70% ethanol in heptane at a flow rate of 15 ml/min.

### Enantiomer 1

Analytical Chiral HPLC (25 cm Chiralpak AD column eluting with 70% ethanol in heptane at a flow rate of 1 ml/min.)—Retention time 5.2 mins.

### Enantiomer 2

Analytical Chiral HPLC (25 cm Chiralpak AD column eluting with 70% ethanol in heptane at a flow rate of 1 ml/min.)—Retention time 12 mins.

The following compounds were similarly prepared:

| | | | Retention time/min | |
|---|---|---|---|---|
| Example | | | Chiral | |
| Number | Structure | Name | HPLC* | LCMS |
| 6 | 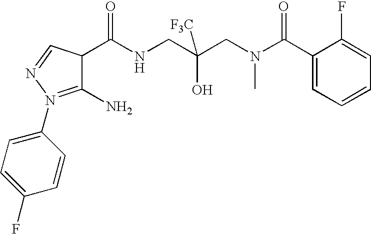 | 5-amino-1-(4- fluorophenyl)-N- (3,3,3-trifluoro-2- {[[(2- fluorophenyl)car- bonyl](methyl)amino] methyl}-2- hydroxypropyl)-1H- pyrazole-4- carboxamide | — | 3.06 |
| 7 (Rac) E1 E2 | 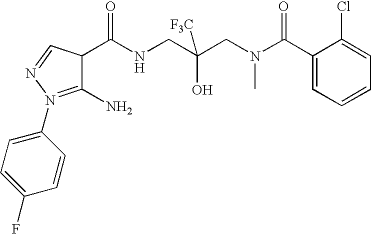 | 5-amino-N-(2-{[[(2- chlorophenyl)car- bonyl](methyl)amino] methyl}-3,3,3- trifluoro-2- hydroxypropyl)-1- (4-fluorophenyl)- 1H- pyrazole-4- carboxamide | — 7.4 15.8 (Chiralpak AD 60% EtOH/ heptane) | 3.13 |
| 8 (Rac) E1 E2 | 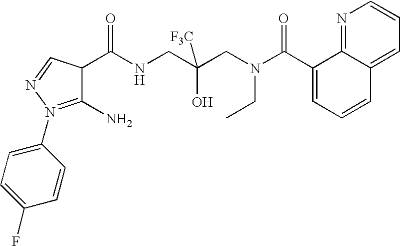 | N-{2-[({[5-amino-1- (4-fluorophenyl)- 1H-pyrazol-4- yl]carbonyl}amino) methyl]-3,3,3- trifluoro-2-hydroxy propyl}-N-ethyl-8- quinolinecarboxamide | — 4.5 10.3 (Chiralcel OD 70% EtOH/ heptane) | 3.14 |
| 9 (Rac) E1 E2 | 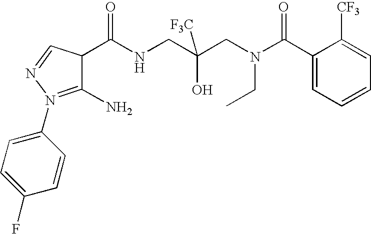 | 5-amino-N-{2- [(ethyl{[2- (trifluoromethyl) phenyl]carbonyl}amino) methyl]-3,3,3- trifluoro-2- hydroxypropyl}-1- (4-fluorophenyl)- 1H- pyrazole-4- carboxamide | — 3.6 6.1 (Chiralcel OD 50% EtOH/ heptane) | 3.35 |
| 10 | 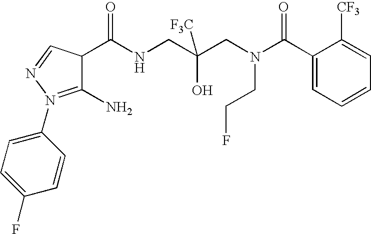 | 5-amino-1-(4- fluorophenyl)-N- {3,3,3-trifluoro-2- [((2-fluoroethyl){[2- (trifluoromethyl) phenyl]carbonyl}amino) methyl]-2- hydroxypropyl}-1H- pyrazole-4- carboxamide | — | 3.34 |
| 11 | 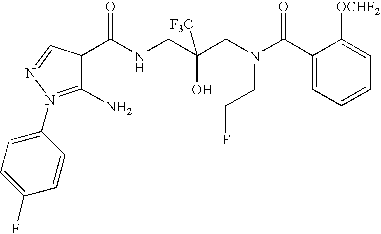 | 5-amino-N-(2-{[({2- [(difluoromethyl) oxy]phenyl}carbonyl) (2-fluoroethyl)amino] methyl}-3,3,3- trifluoro-2- hydroxypropyl)-1- (4-fluorophenyl)- 1H-pyrazole-4- carboxamide | — | 3.26 |
| 12 | 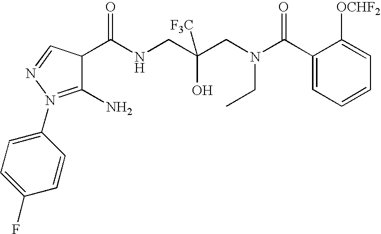 | 5-amino-N-(2-{[({2- [(difluoromethyl) oxy]phenyl}carbonyl) (ethyl)amino] methyl}-3,3,3- trifluoro-2- hydroxypropyl)-1- (4-fluorophenyl)- 1H-pyrazole-4- carboxamide | — | 3.29 |
| 13 | 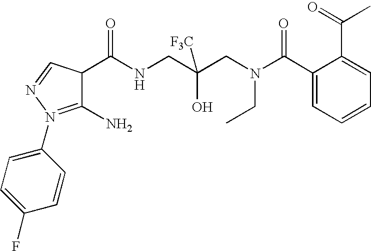 | N-(2-{[[(2- acetylphenyl)car- bonyl](ethyl)amino] methyl}-3,3,3- trifluoro-2- hydroxypropyl)-5- amino-1-(4- fluorophenyl)-1H- pyrazole-4- carboxamide | — | 3.06 |
| 14 (Rac) E1 E2 | 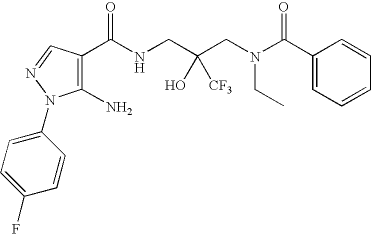 | 5-amino-N-(2- {[ethyl(phenylcar- bonyl)amino]methyl}- 3,3,3-trifluoro-2- hydroxypropyl)-1- (4-fluorophenyl)- 1H-pyrazole-4- carboxamide | — 13.1 16.5 (Chiralpak AD 40% EtOH/ heptane) | 3.30 |
| 15 (Rac) E1 E2 | 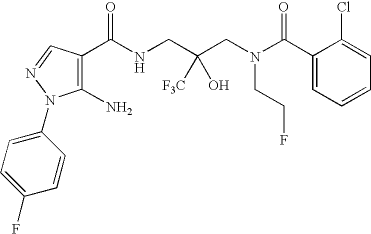 | 5-amino-N-(2-{[[(2- chlorophenyl)car- bonyl](2- fluoroethyl)amino] methyl}-3,3,3- trifluoro-2- hydroxypropyl)-1- (4-fluorophenyl)- 1H-pyrazole-4- carboxamide | — 4.8 8.0 (Chiralcel OD 50% EtOH/ heptane) | 3.15 |
| 16 (Rac) E1 E2 | 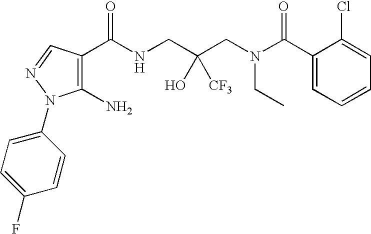 | 5-amino-N-(2-{[[(2- chlorophenyl)car- bonyl](ethyl)amino] methyl}-3,3,3- trifluoro-2- hydroxypropyl)-1- (4-fluorophenyl)- 1H-pyrazole-4- carboxamide | — 10.2 19.5 (Chiralpak AD 40% EtOH/ heptane) | 3.30 |
| 17 | 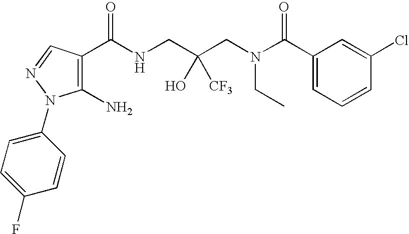 | 5-amino-N-(2-{[[(3- chlorophenyl)car- bonyl](ethyl)amino] methyl}-3,3,3- trifluoro-2- hydroxypropyl)-1- (4-fluorophenyl)- 1H-pyrazole-4- carboxamide | | 3.36 |
| 18 | 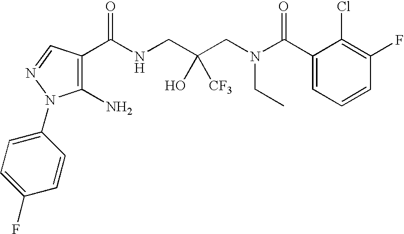 | 5-amino-N-(2-{[[(2- chloro-3- fluorophenyl)car- bonyl](ethyl)amino] methyl}-3,3,3- trifluoro-2- hydroxypropyl)-1- (4-fluorophenyl)- 1H-pyrazole-4- carboxamide | | 3.35 |
| 19 | 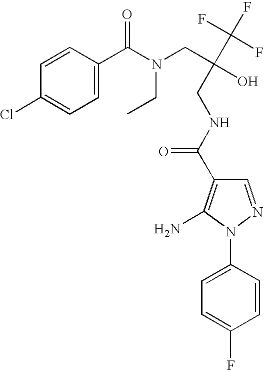 | 5-amino-N-(2-{[[(4- chlorophenyl)car- bonyl](ethyl)amino] methyl}-3,3,3- trifluoro-2- hydroxypropyl)-1- (4-fluorophenyl)- 1H-pyrazole-4- carboxamide | | 3.31 |
| 20 | 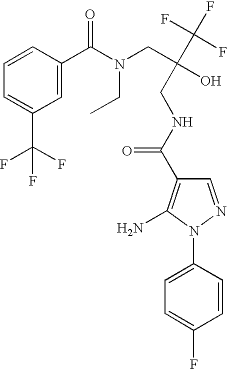 | 5-amino-N-{2- [(ethyl{[3- (trifluoromethyl) phenyl]carbonyl}amino) methyl]-3,3,3- trifluoro-2- hydroxypropyl}-1- (4-fluorophenyl)- 1H-pyrazole-4- carboxamide | | 3.37 |
| 21 | 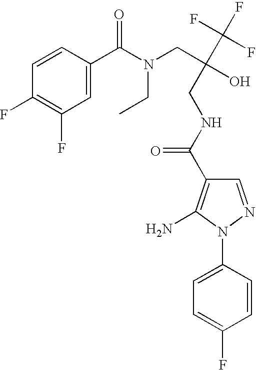 | 5-amino-N-(2- {[[(3,4- difiuorophenyl)car- bonyl](ethyl)amino] methyl}-3,3,3- trifluoro-2- hydroxypropyl)-1- (4-fluorophenyl)- 1H-pyrazole-4- carboxamide | | 3.29 |
| 22 | 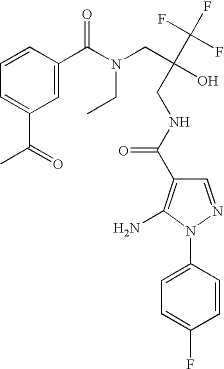 | N-(2-{[[(3- acetylphenyl)car- bonyl](ethyl)amino] methyl}-3,3,3- trifluoro-2- hydroxypropyl)-5- amino-1-(4- fluorophenyl)-1H- pyrazole-4- carboxamide | | 3.08 |
| 23 | 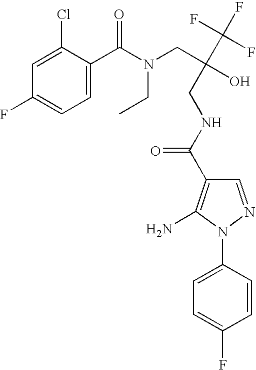 | 5-amino-N-(2-{[[(2- chloro-4- fluorophenyl)car- bonyl](ethyl)amino] methyl}-3,3,3- trifluoro-2- hydroxypropyl)-1- (4-fluorophenyl)- 1H-pyrazole-4- carboxamide | | 3.36 |
| 25 | 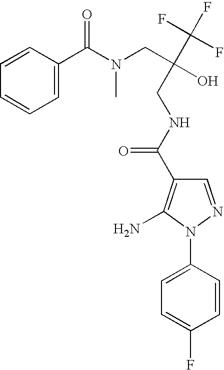 | 5-amino-1-(4- fluorophenyl)-N- (3,3,3-trifluoro-2- hydroxy-2- {[methyl(phenylcar bonyl)amino]methyl} propyl)-1H- pyrazole-4- carboxamide | | 3.02 |
| 26 | 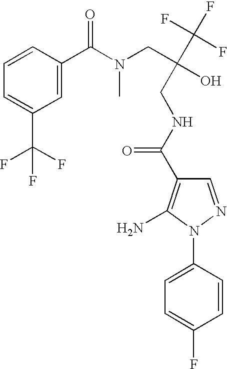 | 5-amino-1-(4- fluorophenyl)-N- {3,3,3-trifluoro-2- hydroxy-2- [(methyl{[3- (trifluoromethyl) phenyl]carbonyl}amino) methyl]propyl}- 1H-pyrazole-4- carboxamide | | 3.26 |
| 27 | 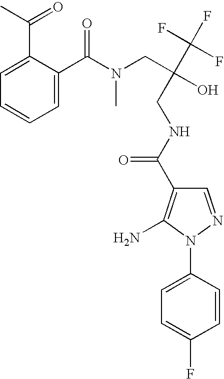 | N-(2-{[[(2- acetylphenyl)car- bonyl](methyl)amino] methyl}-3,3,3- trifluoro-2- hydroxypropyl)-5- amino-1-(4- fluorophenyl)-1H- pyrazole-4- carboxamide | | 2.90 |
| 28 | 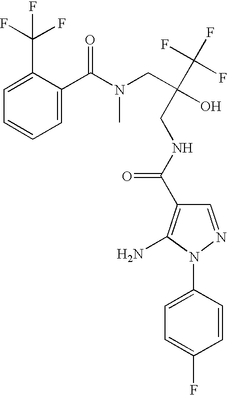 | 5-amino-1-(4- fluorophenyl)-N- {3,3,3-trifluoro-2- hydroxy-2- [(methyl{[2- (trifluoromethyl) phenyl]carbonyl}amino) methyl]propyl}- 1H-pyrazole-4- carboxamide | | 3.28 |
| 29 | 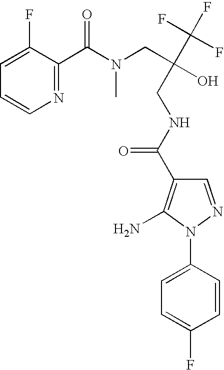 | N-{2-[({[5-amino-1- (4-fluorophenyl)- 1H-pyrazol-4- yl]carbonyl}amino) methyl]-3,3,3- trifluoro-2- hydroxypropyl}-3- fluoro-N-methyl-2- pyridinecarboxamide | | 2.84 |
| 30 | 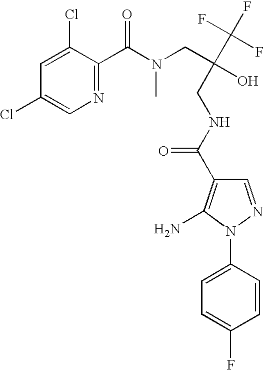 | N-{2-[({[5-amino-1- (4-fluorophenyl)- 1H-pyrazol-4- yl]carbonyl}amino) methyl]-3,3,3- trifluoro-2- hydroxypropyl}- 3,5-dichloro-N- methyl-2- pyridinecarboxamide | | 3.19 |
| 31 | 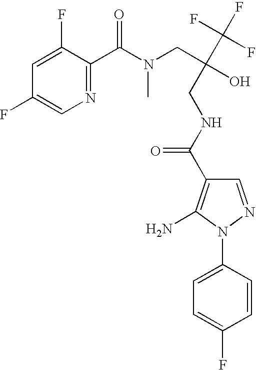 | N-{2-[({[5-amino-1- (4-fluorophenyl)- 1H-pyrazol-4- yl]carbonyl}amino) methyl]-3,3,3- trifluoro-2- hydroxypropyl}- 3,5-difluoro-N- methyl-2- pyridinecarboxamide | | 2.92 |
| 38 | 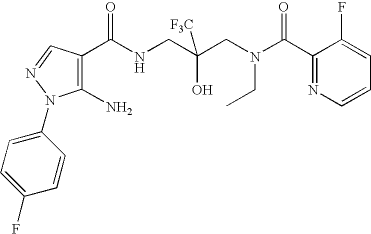 | N-{2-[({[5-amino-1- (4-fluorophenyl)- 1H-pyrazol-4- yl]carbonyl}amino) methyl]-3,3,3- trifluoro-2- hydroxypropyl}-N- ethyl-3-fluoro-2- pyridinecarboxamide | | 3.06 |
| 39 | 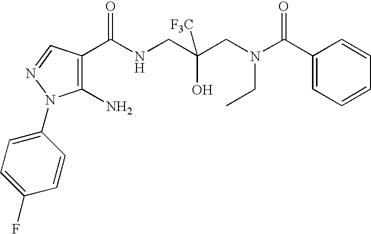 | 5-amino-N-(2- [ethyl(phenylcar- bonyl)amino]methyl}- 3,3,3-trifluoro-2- hydroxypropyl)-1- (4-fluorophenyl)- 1H-pyrazole-4- carboxamide | | 3.30 |
| 40 | 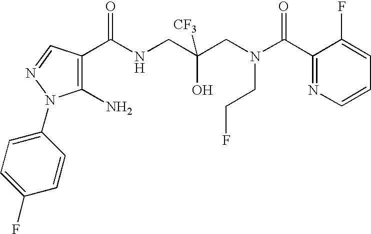 | N-{2-[({[5-amino-1- (4-fluorophenyl)- 1H-pyrazol-4- yl]carbonyl}amino) methyl]-3,3,3- trifluoro-2- hydroxypropyl}-3- fluoro-N-(2- fluoroethyl)-2- pyridinecarboxamide | | 3.04 |
| 41 | 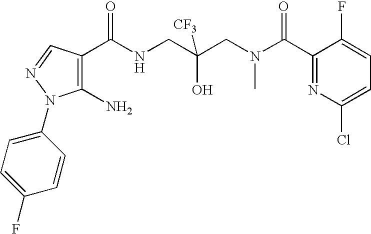 | N-{2-[({[5-amino-1- (4-fluorophenyl)- 1H-pyrazol-4- yl]carbonyl}amino) methyl]-3,3,3- trifluoro-2- hydroxypropyl}-6- chloro-3-fluoro-N- methyl-2- pyridinecarboxamide | | 3.16 |
| 42 | 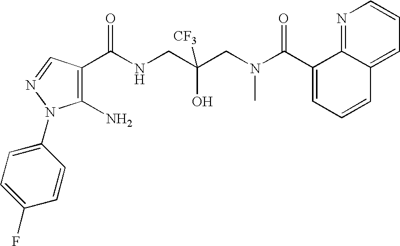 | N-{2-[({[5-amino-1- (4-fluorophenyl)- 1H-pyrazol-4- yl]carbonyl}amino) methyl]-3,3,3- trifluoro-2- hydroxypropyl}-N- methyl-8- quinolinecarboxamide | | 3.00 |
| 43 | 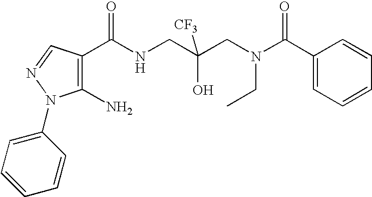 | 5-amino-N-(2- {[ethyl(phenylcar- bonyl)amino]methyl}- 3,3,3-trifluoro-2- hydroxypropyl)-1- phenyl-1H- pyrazole-4- carboxamide | | 3.07 |
| 44 | 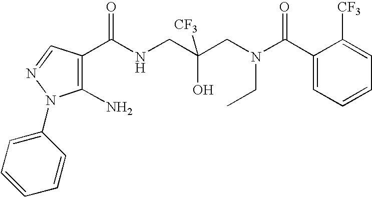 | 5-amino-N-{2- [(ethyl{[2- (trifluoromethyl) phenyl]carbonyl}amino) methyl]-3,3,3- trifluoro-2- hydroxypropyl}-1- phenyl-1H- pyrazole-4- carboxamide | | 3.31 |
| 45 (Rac) E1 E2 | 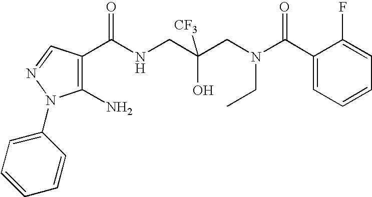 | 5-amino-N-[2- ({ethyl[(2- fluorophenyl)car- bonyl]amino}methyl)- 3,3,3-trifluoro-2- hydroxypropyl]-1- phenyl-1H- pyrazole-4- carboxamide | — 5.7 11.5 (Chiralcel OD 50% EtOH/ heptane) | 3.18 |
| 46 | 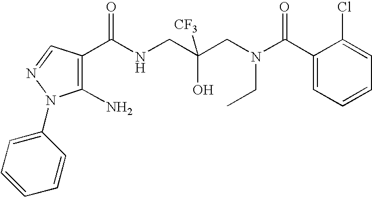 | 5-amino-N-(2-{[[(2- chlorophenyl)car- bonyl](ethyl)amino] methyl}-3,3,3- trifluoro-2- hydroxypropyl)-1- phenyl-1H- pyrazole-4- carboxamide | | 3.2 |

*All chiral analytical columns were 25 cm and the flow rate was 1 ml/min

The following compounds were prepared in an array as described below

| | | | Retention |
|---|---|---|---|
| | | | Time/min |
| Example | Structure | Name | LCMS |
| 24 | 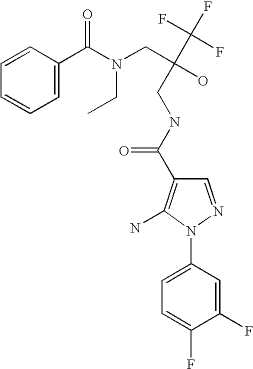 | 5-amino-1-(3,4-difluorophenyl)- N-(2- {[ethyl(phenylcarbonyl)amino] methyl}-3,3,3-trifluoro-2- hydroxypropyl)-1H-pyrazole-4- carboxamide | 2.63 |
| 32 | 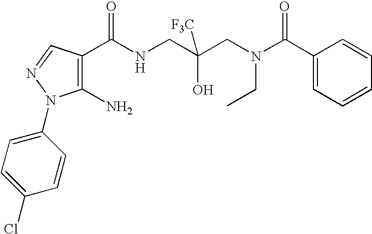 | 5-amino-1-(4-chlorophenyl)-N- (2-{[ethyl(phenylcarbonyl)amino] methyl}-3,3,3-trifluoro-2- hydroxypropyl)-1H-pyrazole-4- carboxamide | 2.98 |
| 33 | 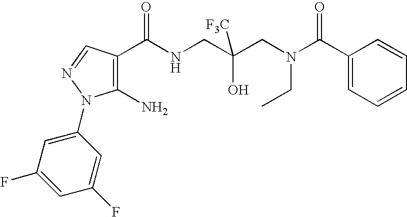 | 5-amino-1-(3,5-difluorophenyl)- N-(2-{[ethyl(phenylcarbonyl)amino] methyl}-3,3,3-trifluoro-2- hydroxypropyl)-1H-pyrazole-4- carboxamide | 2.81 |
| 34 | 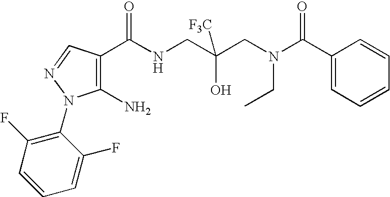 | N-{2-[({1-[5-amino-1-(2,6- difluorophenyl)-1H-pyrazol-4- yl]ethenyl}amino)methyl]-3,3,3- trifluoro-2-hydroxypropyl}-N- ethylbenzamide | 2.23 |
| 35 | 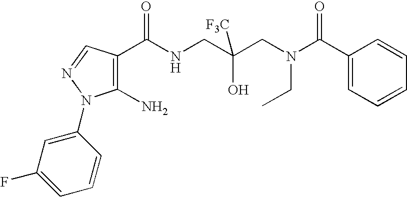 | 5-amino-N-(2- {[ethyl(phenylcarbonyl)amino] methyl}-3,3,3-trifluoro-2- hydroxypropyl)-1-(3- fluorophenyl)-1H-pyrazole-4- carboxamide | 2.58 |

A solution of O-(7-azabenzotriazol-1-yl)-N,N,N′,N′-tetramethyluronium hexafluorophosphate (HATU) (0.54 mmol) in N,N-dimethylformamide (0.2 ml) was added to each of the acids (0.9 mmol) shown in the table below. N,N-diisopropylethylamine (0.1 ml) was added and after 5 mins the solutions were added to N-[2-(aminomethyl)-3,3,3-trifluoro-2-hydroxypropyl]-N-ethylbenzamide hydrochloride (0.69 mmol) and left overnight. The products were isolated and purified as in Example 5.

| Example | Acid |
|---|---|
| 24 | 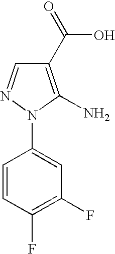 |
| 32 | 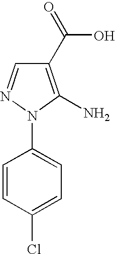 |
| 33 | 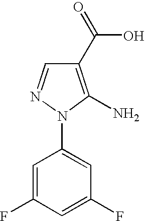 |
| 34 | 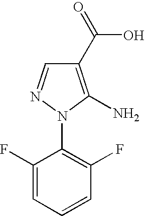 |
| 35 | 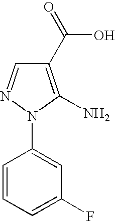 |

The following compounds were similarly prepared:

| | | | Retention |
|---|---|---|---|
| | | | time/min |
| Example | Structure | Name | LCMS |
| 36 | 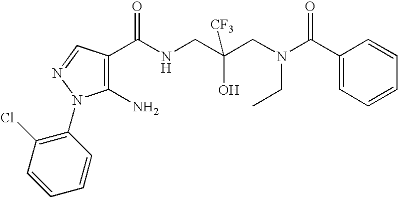 | 5-amino-1-(2- chlorophenyl)-N-(2- {[ethyl(phenylcar- bonyl)amino]methyl}- 3,3,3-trifluoro-2- hydroxypropyl)-1H- pyrazole-4- carboxamide | 3.13 |
| 37 | 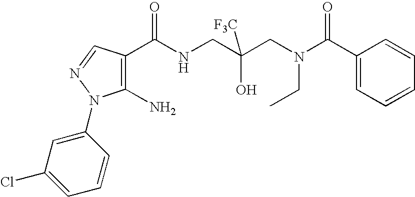 | 5-amino-1-(3- chlorophenyl)-N-(2- {[ethyl(phenylcar- bonyl)amino]methyl}- 3,3,3-trifluoro-2- hydroxypropyl)-1H- pyrazole-4- carboxamide | 3.33 |

using the following acids:

| Example | Acid |
|---|---|
| 36 | 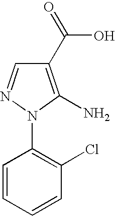 |
| 37 | 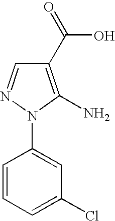 |

### BIOLOGICAL EXAMPLES

### Glucocorticoid Receptor Binding Assay

The ability of compounds to bind to the glucocorticoid receptor was determined by assessing their ability to compete with an Alexa 555 fluorescently-labelled dexamethasone derivative. Compounds were solvated and diluted in DMSO, and transferred directly into assay plates. Fluorescent dexamethasone and a partially purified full length glucocorticoid receptor were added to the plates, together with buffer components to stabilise the GR protein and incubated at room temperature for 2 hr in the dark. Binding of each compound was assessed by analysing the displacement of fluorescent ligand by measuring the decrease in fluorescence polarisation signal from the mixture.

Example 1 (racemic), Example 1 Enantiomer 1, Example 1 Enantiomer 2, Example 2 (racemic), Example 2 Enantiomer 1, Example 2 Enantiomer 2, Example 3 (racemic), Example 4 (racemic), Example 4 Enantiomer 1, Example 4 Enantiomer 2, Example 5 (racemic), Example 5 Enantiomer 1, Example 5 Enantiomer 2, Example 6 (racemic), Example 7 (racemic), Example 7 Enantiomer 1, Example 7 Enantiomer 2, Example 8 Enantiomer 1, Example 8 Enantiomer 2, Example 9 (racemic), Example 9 Enantiomer 1, Example 9 Enantiomer 2, Example 10 (racemic), Example 12 (racemic), Example 13 (racemic), Example 14 (racemic), Example 14 Enantiomer 1, Example 14 Enantiomer 2, Example 15 (racemic), Example 16 (racemic), Example 17 (racemic), Example 18 (racemic), Example 19 (racemic), Example 20 (racemic), Example 21 (racemic), Example 22 (racemic), Example 23 (racemic), Example 24 (racemic), Example 25 (racemic), Example 26 (racemic), Example 27 (racemic), Example 28 (racemic), Example 32 (racemic), Example 33 (racemic), Example 34 (racemic), Example 35 (racemic), Example 36 (racemic), Example 37 (racemic), Example 38 (racemic), Example 39 (racemic), Example 40 (racemic), Example 43 (racemic), Example 45 (racemic), Example 45 Enantiomer 1, Example 45 Enantiomer 2 and Example 46 (racemic) have shown glucocorticoid binding with a pIC₅₀>6 at least once in this assay.

### A549 SPAP Cells

Human Caucasian lung carcinoma A549 cell line (ECACC No. 86012804) has been stably transfected in house with a plasmid containing an ELAM promoter sequence that has a NFκB response element within it. Stimulation of the cell line with TNFα results in intracellular signal transduction and ultimately translocation of NFκB into the nucleus. This activates the inserted DNA sequence resulting in transcription of the integrated SPAP gene, which is quantified using a calorimetric assay. In this assay, GR agonist compounds inhibit NFκB driven transcription resulting in a decrease in signal. The stably transfected cell line was grown as a monolayer in DMEM supplemented with FCS-HI (10%), Non-essential amino acids (1%), L-Glutamine (2 mM), Pen/Strep (1%) and Geneticin (50 mg/ml).

### GR Functional Assay —NFκB Assay

A 70% confluent T225 flask of A549 SPAP cells was harvested by centrifugation for 5 mins at 200 g, resuspended in assay buffer (DMEM supplemented with 10% FCS 2×HI, 2 mM L-Glutamine, 1% Pen/Strep and Non essential amino acids) and diluted to 0.16×10⁶/ml. 60 μl of cell solution was dispensed to each well of clear Nunc 384-well plates, containing compound at the required concentration. Plates were incubated for 1 h at 37° C., 95% humidity, 5% CO₂ before 10 μl of TNFα was added at final concentration of 3.2 ng/ml and then returned to the cell incubator for 15 hr. Plates were equilibrated to room temperature for 1 hr prior to the addition of 25 μl of pNPP buffer (1M Diethanolamine pH 9.8, 0.5 mM MgCl₂, 0.28M NaCl, 2 mg/ml pNPP) to each well of assay plates. The plates were covered to protect the reagents from light, and then incubated at room temperature for approximately 1 hr before reading them on an Ascent using a 405 nm single filter.

Example 1 (racemic), Example 1 Enantiomer 1, Example 1 Enantiomer 2, Example 2 (racemic), Example 2 Enantiomer 1, Example 2 Enantiomer 2, Example 3 (racemic), Example 3 Enantiomer 1, Example 3 Enantiomer 2, Example 4 (racemic), Example 4 Enantiomer 1, Example 4 Enantiomer 2, Example 5 (racemic), Example 5 Enantiomer 1, Example 5 Enantiomer 2, Example 6 (racemic), Example 7 (racemic), Example 7 Enantiomer 1, Example 8 (racemic), Example 8 Enantiomer 1, Example 8 Enantiomer 2, Example 9 (racemic), Example 9 Enantiomer 1, Example 9 Enantiomer 2, Examples 10 to 13 (racemic), Example 14 (racemic), Example 14 Enantiomer 2, Example 15 (racemic), Example 16 (racemic), Example 16 Enantiomer 1, Example 16 Enantiomer 2, Example 17 (racemic), Example 18 (racemic), Example 20 (racemic), Example 21 (racemic), Example 23 (racemic), Example 24 (racemic), Example 25 (racemic), Example 27 (racemic), Example 28 (racemic), Example 29 (racemic) Examples 32 to 44 (racemic), Example 45 (racemic), Example 45 Enantiomer 1, Example 45 Enantiomer 2, and Example 46 have shown pIC₅₀ values of >6.0 at least once in the NFkB assay.

### A549 MMTV Cells

Human Caucasian lung carcinoma A549 cell line (ECACC No. 86012804) has been stably transfected in house with a plasmid containing a renilla luciferase reporter with an MMTV promoter. Stimulation of the cell line with GR agonists results in intracellular signal transduction and ultimately translocation of GR into the nucleus. This activates the inserted DNA sequence resulting in transcription of the integrated luciferase gene, which is quantified using a light emission. The stably transfected cell line was grown as a monolayer in DMEM supplemented with FCS-HI (10%), Non-essential amino acids (1%), L-Glutamine (2 mM), Pen/Strep (1%) and Geneticin (50 mg/ml).

### GR Agonist Assay—MMTV Assay

A 90% confluent T175 flask of A549 MMTV cells was harvested by centrifugation for 5 min at 200 g, resuspended in assay buffer (DMEM supplemented with 10% FCS 2×HI, 2 mM Glutamax, Non essential amino acids and 25 mM HEPES) and diluted to 0.1×10⁶/ml. 70 μl of cell solution was dispensed to each well of white Nunc 384-well plates, containing compound at the required concentration. Plates were incubated for 6 hr at 37° C., 95% humidity, 5% CO₂. Plates were equilibrated to room temperature for 1 hr prior to the addition of 101 of Renilla substrate to each well of assay plates. The plates were covered to protect the reagents from light, and then incubated at room temperature for approximately 15 mins before reading them on a Viewlux.

The following examples are agonists (i.e. have an average maximum asymptote of ≧40%) in the NFkB and MMTV agonist assays and have shown potency of pIC₅₀>6.5 at least once in the NFKB assay:

Example 1 (racemic), Example 1 Enantiomer 2, Example 2 (racemic), Example 2 Enantiomer 1, Example 2 Enantiomer 2, Example 3 (racemic), Example 3 Enantiomer 1, Example 4 (racemic), Example 4 Enantiomer 1, Example 4 Enantiomer 2, Example 5 (racemic), Example 5 Enantiomer 1, Example 6 (racemic), Example 7 (racemic), Example 7 Enantiomer 1, Example 8 (racemic), Example 8 Enantiomer 1, Example 9 (racemic), Example 9 Enantiomer 1, Example 9 Enantiomer 2, Example 10 (racemic), Example 11 (racemic), Example 12 (racemic), Example 13 (racemic), Example 14 (racemic), Example 14 Enantiomer 2, Example 15 (racemic), Example 16 (racemic), Example 16 Enantiomer 1, Example 16 Enantiomer 2, Example 17 (racemic), Example 18 (racemic), Example 20 (racemic), Example 23 (racemic), Example 24 (racemic), Example 27 (racemic), Example 28 (racemic), Example 32 (racemic), Example 33 (racemic), Example 34 (racemic), Example 35 (racemic), Example 37 (racemic), Example 38 (racemic), Example 39 (racemic), Example 40 (racemic), Example 42 (racemic), Example 43 (racemic), Example 45 (racemic), Example 45 Enantiomer 1, Example 45 Enantiomer 2 and Example 46 (racemic).

### Assay for Progesterone Receptor Agonist Activity

A T225 flask of CV-1 cells at a density of 80% confluency was washed with PBS, detached from the flask using 0.25% trypsin and counted using a Sysmex KX-21N. Cells were diluted in DMEM containing 10% Hyclone, 2 mM L-Glutamate and 1% Pen/Strep at 140 cells/μl and transduced with 10% PRb-BacMam and 10% MMTV-BacMam. 70 ml of suspension cells were dispensed to each well of white Nunc 384-well plates, containing compounds at the required concentration. After 24 hr 10 μl of Steadylite were added to each well of the plates. Plates were incubated in the dark for 10 min before reading them on a Viewlux reader. Dose response curves were constructed from which pEC₅₀ values were estimated.

Example 1 (racemic), Example 1 Enantiomer 1, Example 1 Enantiomer 2, Example 2 (racemic), Example 2 Enantiomer 1, Example 2 Enantiomer 2, Example 3 (racemic), Example 3 Enantiomer 1, Example 4 (racemic), Example 4 Enantiomer 1, Example 4 Enantiomer 2, Example 5 (racemic), Example 5 Enantiomer 1, Example 5 Enantiomer 2, Example 6 (racemic). Example 7 (racemic), Example 7 Enantiomer 1, Example 7 Enantiomer 2, Example 8 (racemic), Example 8 Enantiomer 1, Example 8 Enantiomer 2, Example 9 (racemic), Example 9 Enantiomer 1, Example 9 Enantiomer 2, Examples 11 to 13 (racemic), Example 14 (racemic), Example 14 Enantiomer 1, Example 14 Enantiomer 2, Example 15 (racemic), Example 16 (racemic), Example 16 Enantiomer 1, Example 16 Enantiomer 2, Examples 17 to 28 (racemic), Examples 32 to 36 (racemic), Examples 38 to 40 (racemic), Examples 42 to 44 (racemic), Example 45 (racemic), Example 45 Enantiomer 1, Example 45 Enantiomer 2 and Example 46 have shown pEC₅₀<6 at least once in this assay.

### Assay for Brain Penetrance

Each rat received a single intravenous dose at a level of 1 mg/kg. The dose was formulated in 10% DMSO/50% PEG200/40% sterile water. Terminal blood samples were taken at 5 or 15 mins after dosing, by cardiac puncture following anaesthesia with isofluorane. The brains were removed at the same time point.

The compounds were extracted from 20 μL plasma by protein precipitation using 120 μL acetonitrile containing an analogue compound as an internal standard. The filtered extracts were collected into a 96 well plate and were diluted with an equal volume of 10% acetonitrile containing 0.1% formic acid in water (v/v). The plate was then mixed on a plate shaker for at least 5 mins before analysis by LC-MS/MS against a calibration line prepared in control plasma.

Each brain was weighed, then homogenised in 3 ml acetonitrile:water (10:90v/v). The compounds were extracted from 200 μL of the resulting homogenate by protein precipitation using 600 μL acetonitrile containing an analogue compound as an internal standard. The extracts were centrifuged and 150 μl of each was filtered and transferred to a 96 well plate. The aliquot was diluted with 10% acetonitrile containing 0.1% formic acid in water (v/v). The plate was mixed on a plate shaker for at least 5 mins before analysis by LC-MS/MS against a calibration line prepared in control brain homogenate.

In this assay, Example 1 (racemic), Example 2 (racemic), Example 3 Enantiomer 1, Example 4 (racemic), Example 5 (racemic) and Example 24 (racemic) have shown a brain to plasma ratio equal to or greater than 0.1 at 5 mins in this assay.

In describing examples according to their activity in the assays above, it will be appreciated that at least one isomer, for example, an enantiomer in a mixture of isomers (such as a racemate) has the described activity. The other enantiomer may have similar activity, less activity, no activity or may have some antagonist activity in the case of a functional assay.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

The patents and patent applications described in this application are herein incorporated by reference.

What is claimed is:

1. A compound of formula (I): 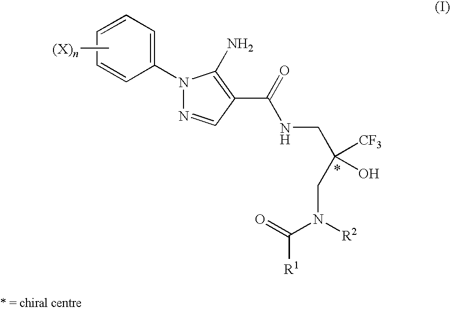
wherein
is selected from the group consisting of 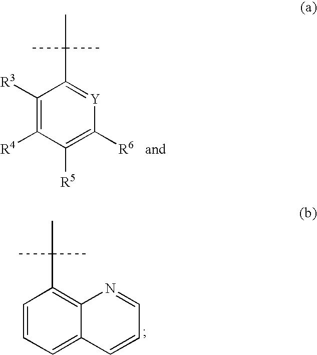
R² is selected from the group consisting of methyl, ethyl and 2-fluoroethyl;
R³, R⁴, R⁵ and R⁶ are each independently selected from the group consisting of hydrogen, fluorine, chlorine, —CF₃, —CHF₂, —OCHF₂ and —C(O)CH₃;
Y is selected from the group consisting of nitrogen and CH;
n is an integer selected from the group consisting of 0, 1 and 2,
when n is 1, X is selected from the group consisting of chlorine and fluorine, and
when n is 2, each X is fluorine;
or a salt thereof.

2. A compound according to claim 1 wherein R¹ is

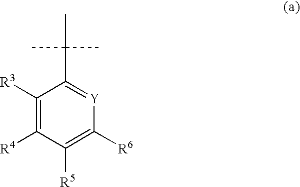

3. A compound according to claim 1, wherein R³ is selected from the group consisting of hydrogen, fluorine and chlorine.

4. A compound according to claim 1 wherein R⁴ is selected from the group consisting of hydrogen and fluorine.

5. A compound according to claim 1 wherein n is 1.

6. A compound according to claim 5 wherein X is fluorine in the para position on the phenyl ring.

7. A compound which is selected from the group consisting of:
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide;
5-amino-1-(4-fluorophenyl)-N-(3,3,3-trifluoro-2-{[(2-fluoroethyl)(phenylcarbonyl)amino]methyl}-2-hydroxypropyl)-1H-pyrazole-4-carboxamide (Enantiomer 2);
5-amino-1-(4-fluorophenyl)-N-[3,3,3-trifluoro-2-({(2-fluoroethyl)[(2-fluorophenyl)carbonyl]amino}methyl)-2-hydroxypropyl]-1H-pyrazole-4-carboxamide;
5-amino-1-(4-fluorophenyl)-N-[3,3,3-trifluoro-2-({(2-fluoroethyl)[(2-fluorophenyl)carbonyl]amino}methyl)-2-hydroxypropyl]-1H-pyrazole-4-carboxamide (Enantiomer 2);
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide;
N-{2-[({[5-amino-1-(4-fluorophenyl)-1H-pyrazol-4-yl]carbonyl}amino)methyl]-3,3,3-trifluoro-2-hydroxypropyl}-3-chloro-N-ethyl-2-pyridinecarboxamide (Enantiomer 1);
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide;
5-amino-N-[2-({ethyl[(2-fluorophenyl)carbonyl]amino}methyl)-3,3,3-trifluoro-2-hydroxypropyl]-1-(4-fluorophenyl)-1H-pyrazole-4-carboxamide (Enantiomer 2);
5-amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(2,3-difluorophenyl)carbonyl](ethyl)amino]methyl}-3,3,3-trifluoro-2-hydroxypropyl)-1-(4-fluorophenyl)-1H-pyrazole-4-carboxamide (Enantiomer 1);
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide;
5-amino-N-(2-{[[(2-chlorophenyl)carbonyl](methyl)amino]methyl}-3,3,3-trifluoro-2-hydroxypropyl)-1-(4-fluorophenyl)-1H-pyrazole-4-carboxamide (Enantiomer 1);
N-{2-[({[5-amino-1-(4-fluorophenyl)-1H-pyrazol-4-yl]carbonyl}amino)methyl]-3,3,3-trifluoro-2-hydroxypropyl}-N-ethyl-8-quinolinecarboxamide;
N-{2-[({[5-amino-1-(4-fluorophenyl)-1H-pyrazol-4-yl]carbonyl}amino)methyl]-3,3,3-trifluoro-2-hydroxypropyl}-N-ethyl-8-quinolinecarboxamide (Enantiomer 1);
5-amino-N-(2-{[[(2-chlorophenyl)carbonyl](ethyl)amino]methyl}-3,3,3-trifluoro-2-hydroxypropyl)-1-(4-fluorophenyl)-1H-pyrazole-4-carboxamide;
5-amino-N-(2-{[[(2-chlorophenyl)carbonyl](ethyl)amino]methyl}-3,3,3-trifluoro-2-hydroxypropyl)-1-(4-fluorophenyl)-1H-pyrazole-4-carboxamide (Enantiomer 1);
5-amino-1-(3,4-difluorophenyl)-N-(2-{[ethyl(phenylcarbonyl)amino]methyl}-3,3,3-trifluoro-2-hydroxypropyl)-1H-pyrazole-4-carboxamide;
and salts thereof.

8. (canceled)

9. A method for the treatment of a human or animal subject with an inflammatory and/or autoimmune condition, which method comprises administering to said human or animal subject an effective amount of a compound as claimed in claim 1, or a pharmaceutically acceptable salt thereof.

10. A method for the treatment of a human or animal subject with multiple sclerosis, cerebral vasculitis, neurosarcoidosis, Sjogren's syndrome, systemic lupus erythematosis, acute or chronic inflammatory polyradiculopathy, Alzheimer's disease, neoplastic diseases of the nervous system, trauma or infectious diseases of the nervous system, or brain injury, which method comprises administering to said human or animal subject an effective amount of a compound as claimed in claim 1, or a pharmaceutically acceptable salt thereof.

11. A pharmaceutical composition comprising a compound of formula (I) as claimed in claim 1, or a pharmaceutically acceptable salt thereof, in admixture with one or more physiologically acceptable diluents or carriers.

12. A compound of formula (XV) 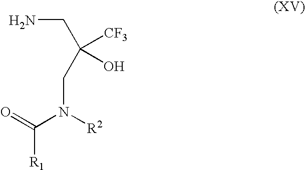
wherein
R¹ is selected from the group consisting of 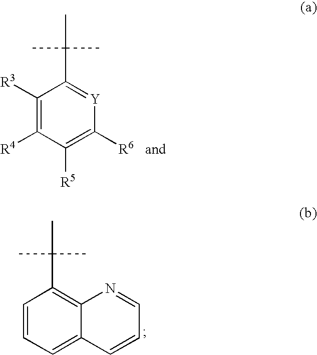
R² is selected from the group consisting of methyl, ethyl and 2-fluoroethyl.

13. A compound of formula (XVI) 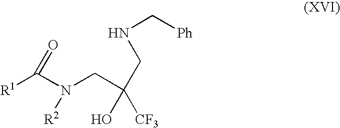
wherein
R¹ is selected from the group consisting of 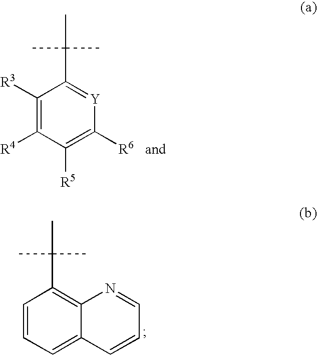
R² is selected from the group consisting of methyl, ethyl and 2-fluoroethyl;
and Ph is phenyl.

14. A compound of formula (XVII) 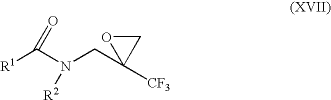
wherein
R¹ is selected from the group consisting of 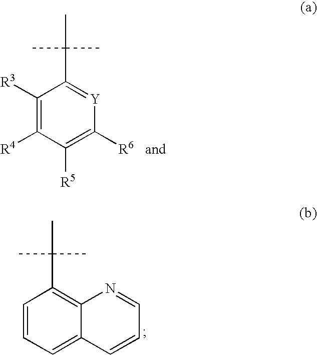
R² is selected from the group consisting of methyl, ethyl and 2-fluoroethyl.
